(19) United States
(12) Patent Application Publication
(10) Pub. No.: US 2025/0201342 A1
(43) Pub. Date: Jun. 19, 2025

(54) MINIMAL RESIDUAL DISEASE (MRD) MODELS FOR DETERMINING LIKELIHOODS OR PROBABILITIES OF A SUBJECT COMPRISING CANCER

(71) Applicant: Illumina, Inc., San Diego, CA (US)

(72) Inventors: Konrad Haarhoff Scheffler, Cambridge (GB); Sven Bilke, Lemon Grove, CA (US); Li Liu, San Diego, CA (US)

(21) Appl. No.: 18/981,285

(22) Filed: Dec. 13, 2024

Related U.S. Application Data
(60) Provisional application No. 63/653,945, filed on May 30, 2024.
(60) Provisional application No. 63/625,830, filed on Jan. 26, 2024.
(60) Provisional application No. 63/611,005, filed on Dec. 15, 2023.

(51) Int. Cl. G16B 20/20; G16B 20/10; G16H 50/20

(52) U.S. Cl. CPC G16B 20/20; G16B 20/10; G16H 50/20

(57) ABSTRACT

The disclosure describes methods, non-transitory computer-readable media, and systems that can (a) compare variants of a tumor profile generated from a subject's tumor sample with variants detected in reads of the subject's subsequent sample and (b) determine a likelihood that the subject's subsequent sample comprises residual tumor material based on the comparison of variants. For example, the disclosed system identifies a tumor profile for a sample with a subset of variants making up the profile. By later sequencing a subsequent sample from the subject and counting biological observables—such as reads supporting the subset of variants in the tumor profile—the system can use a minimal residual disease (MRD) model to compare the variants from the biological observables and the tumor profile's variants to determine a likelihood that the subsequent sample comprises residual tumor material.

### CROSS-REFERENCE TO RELATED APPLICATIONS

This application claims priority to and the benefit of U.S. Provisional Patent Application No. 63/653,945, entitled, “MINIMAL RESIDUAL DISEASE (MRD) MODELS FOR DETERMINING LIKELIHOODS OR PROBABILITIES OF A SUBJECT COMPRISING CANCER,” filed on May 30, 2024 (IP-2750-PRV3); U.S. Provisional Patent Application No. 63/625,830, entitled, “MINIMAL RESIDUAL DISEASE (MRD) MODELS FOR DETERMINING LIKELIHOODS OR PROBABILITIES OF A SUBJECT COMPRISING CANCER,” filed on Jan. 26, 2024 (IP-2750-PRV2); U.S. Provisional Patent Application No. 63/611,005, entitled, “MINIMAL RESIDUAL DISEASE (MRD) MODELS FOR DETERMINING LIKELIHOODS OR PROBABILITIES OF A SUBJECT COMPRISING CANCER,” filed on Dec. 15, 2023 (IP-2750-PRV). Each of the aforementioned applications is hereby incorporated by reference in its entirety.

### BACKGROUND

In recent years, biotechnology firms and research institutions have improved hardware and software platforms for determining various characteristics of a genomic sample or other nucleic-acid polymer. For instance, platforms have been developed for analyzing a nucleotide sequence from a sample of interest (e.g., a genomic sample) and identifying the nucleobases of genes or other genomic regions through a targeted assay—such as by using a sequencing process performed via conventional Sanger sequencing or sequencing-by-synthesis (SBS) for a targeted assay. Through such a process, existing platforms can create one or more nucleotide reads that indicate the sequence of nucleobases contained in the nucleotide sequences of genes or other target genomic regions. Some existing platforms use these nucleotide reads to make further determinations about the sample of interest. For example, some existing assay systems use the nucleotide reads for a targeted assay to detect or monitor minimal residual disease (MRD)—including the presence of residual tumor or cancer cells—in post-treatment cancer patients.

Despite these advances, existing MRD assay systems suffer from technical shortcomings that require relatively higher input of deoxyribonucleic acid (DNA) sample material to achieve relatively higher read depths relative to other assays and result in inflexible and sometimes inaccurate operation while providing tumor analysis. For instance, many existing MRD assay systems require relatively more cell-free DNA (cfDNA) in terms of plasma volume or cfDNA mass from a sample to process sufficient genetic material in a sequencing device to ensure that the sequencing device produces nucleotide reads that map to genes or target genomic regions at relative higher depth (e.g., 120× coverage) relative to other assays. Some such MRD assays use bespoke-based oligonucleotide probes that target a sample's DNA for particular genes or regions and require relatively higher plasma volumes or mass of cfDNA to create cfDNA-based library templates resulting from DNA fragments identified by the bespoke probes to both create a tumor-specific profile and later improve accuracy of MRD detection. After creating a tumor-specific profile for a subject, existing bespoke-based oligonucleotide probes target DNA fragments that may include tumor-specific somatic variants previously identified from a subject's tumor tissue or baseline pre-surgery plasma as part of the tumor-specific profile. But the relatively higher DNA input required by bespoke-probe-based assays significantly limits the number of samples eligible for MRD detection and requires slow turnaround times for MRD detection relative to other assays.

In addition to the limits of requiring relatively higher DNA input and slower time to MRD results, many existing MRD assay systems inflexibly utilize cancer panels specific to a tumor or cancer, but generic for any given sample, when performing MRD detection. Indeed, some existing MRD assay systems only analyze a predefined set of genes and/or variants associated with a particular tumor, such as a specific set of somatic variants for a specific type of tumor (e.g., breast cancer). Because such cancer panels represent a one-panel-fits-all-patients approach, when new variants associated with the tumor arise, existing MRD assay systems cannot expand testing of such variants without going through laborious measures modifying and updating the cancer panel. By testing predefined variant patterns, these existing systems can miss important variant information regarding the presence of MRD in a subject and fail to adapt quickly (or at all) to new variants present in a given sample with a more diverse or unique set of variants.

Beyond generic cancer panels or other preset panels, some existing MRD assay systems do not accurately account for (or distinguish) noisy nucleotide reads and thereby decrease chances of accurately detecting MRD in a subject. By implementing methods and models that fail to consider or accurately account for the aforementioned noisy nucleotide reads, existing MRD assay systems often provide detection results that exhibit false variants not present in the subject's DNA or exhibit no variants where the subject's DNA indeed includes variants obfuscated by noisy nucleotide reads. To illustrate, by failing to distinguish between noisy reads that resemble circulating-tumor DNA (ctDNA) and actual ctDNA supporting reads, existing MRD assay systems may generate false positive variants for a sample, thereby leading to an inaccurate MRD status.

These, along with additional problems and issues exist in existing MRD assay systems.

### SUMMARY

This disclosure describes embodiments of methods, non-transitory computer readable media, and systems that that can solve one or more of the foregoing (or other problems) in the art. To solve such problems, the disclosed systems that can (a) compare variants of a tumor profile generated from a subject's tumor sample with variants detected in nucleotide reads of the subject's subsequent sample and (b) determine a likelihood that the subject's subsequent sample comprises residual tumor material based on the comparison of variants. To facilitate such a residual-tumor likelihood, for example, the disclosed MRD system identifies a subject-specific tumor profile for a sample with a subset of variants making up the profile. By later sequencing a subsequent sample from the same subject and counting biological observables—such as by determining nucleotide reads supporting the subset of variants in the tumor profile—the MRD system can use an MRD model to compare the variants from the biological observables and the variants in the tumor profile to determine a likelihood that the subsequent sample comprises residual tumor material from the same tumor(s). As set forth further below, the disclosed MRD system thereby improves the accuracy of MRD and detection of residual tumor material across different tumors.

Additional features and advantages of one or more implementations of the present disclosure are outlined in the description which follows, and in part will be obvious from the description, or may be learned by the practice of such example implementations.

### BRIEF DESCRIPTION OF THE DRAWINGS

The detailed description provides one or more implementations with additional specificity and detail through the use of the accompanying drawings, as briefly described below.

FIG. 1 illustrates a diagram of an environment in which a minimal residual disease (MRD) system can operate in accordance with one or more implementations.

FIG. 2 illustrates a pipeline of the MRD system processing initial sample(s) of a subject to determine a tumor profile and processing a subsequent sample of the subject to determine a likelihood of whether the subsequent sample comprises residual tumor material in accordance with one or more implementations.

FIG. 3 illustrates an overview of the MRD system identifying a tumor profile from an initial sample of a subject comprising a tumor and determining a likelihood that a subsequent sample of the subject comprises residual tumor material based on the supporting nucleotide reads for the subsequent sample in accordance with one or more implementations.

FIG. 4 illustrates the MRD system filtering supporting nucleotide reads in accordance with one or more implementations.

FIGS. 5A-5C illustrate the MRD system utilizing an MRD model to determine a likelihood that a subject comprises residual tumor material based on supporting nucleotide reads in accordance with one or more implementations.

FIG. 6 illustrates the MRD system determining the presence of residual tumor material by utilizing a residual tumor material likelihood threshold in accordance with one or more implementations.

FIGS. 7A-7C illustrates the accuracy and sensitivity of the MRD system in accordance with one or more embodiments.

FIG. 8 illustrates a flowchart of a series of acts for determining a haplotype diversity metric in accordance with one or more implementations.

FIG. 9 illustrates a block diagram of an example computing device for implementing one or more implementations of the present disclosure.

### DETAILED DESCRIPTION

This disclosure describes one or more embodiments of a minimal residual disease (MRD) system that can (a) compare variants of a tumor profile generated from a subject's tumor sample with variants detected in nucleotide reads of the subject's subsequent sample (e.g., plasma sample) and (b) utilize an MRD model to determine a likelihood that the subject's subsequent sample comprises residual tumor material based on the comparison of variants. For example, in one or more embodiments, the disclosed MRD system either generates or accesses a previously generated subject-specific tumor profile, such as a tumor profile created from a tissue biopsy. In some cases, the tumor profile includes one or more of somatic single nucleotide variants (SNVs), phased somatic variants, copy number variants (CNVs), or structural variants (SVs) identified from whole genome sequencing (WGS), whole exome sequencing (WES), or a targeted assay. By later sequencing a subsequent sample from the same subject and counting biological observables—such as by identifying nucleotide reads supporting variant calls for variants in the tumor profile—the MRD system can use an MRD model to compare the variants from the biological observables and the variants in the tumor profile to determine a likelihood that the subsequent sample comprises residual tumor material, such as residual material from the same tumor(s) exhibited by the tumor profile.

To illustrate, in one or more embodiments, the MRD system identifies, for a subject and an initial sample comprising tumor cells, a tumor profile comprising a subset of variants within target genomic regions. Later, the MRD system analyzes a subsequent sample of the subject, such as a plasma sample of cell-free DNA (cfDNA). For the subsequent sample of the subject, the MRD system determines a set of nucleotide reads across the target genomic regions. From the set of nucleotide reads for the subsequent sample of the subject, the MRD system further determines supporting nucleotide reads that exhibit, within the target genomic regions, one or more variants of the subset of variants from the tumor profile. Having identified such supporting nucleotide reads, the MRD system further generates, for an MRD model, one or more model parameters indicating a presence of the tumor within the subject. Such an MRD model can be specific to a variant type, such as an SNV-specific MRD model, a CNV-specific MRD model, or an SV-specific MRD model. By utilizing the MRD model and the one or more model parameters, the MRD system further determines a likelihood that the subsequent sample comprises residual tumor material based on the supporting nucleotide reads for the subsequent sample.

As suggested above, in one or more embodiments, the MRD system performs MRD detection using WGS or WES and various samples from the subject. For instance, the MRD system uses WGS or WES to optionally generate nucleotide reads for an initial sample (e.g., a tumor sample) of the subject. Using the nucleotide reads, in some cases, the MRD system generates a tumor profile that includes or indicates a subset of variants at target genomic regions and/or genomic coordinates. Specifically, the MRD system can generate a tumor profile that is unique to the presence of the tumor in the subject. In the alternative to generating a tumor profile, the MRD system accesses or otherwise identifies such a tumor profile previously generated from initial sample(s) of the subject. After generating or identifying a previously generated tumor profile, the MRD system can use WGS or WES to generate nucleotide reads for a subsequent sample (e.g., plasma sample) of the subject. Such a subsequent sample may be taken after the subject receives treatment for the tumor. The MRD system can subsequently determine if one or more of the nucleotides reads for the subsequent sample support the subset of variants from the tumor profile. For example, the MRD system can determine supporting reads for a particular variant by determining that the variant in the subsequent sample aligns with or overlaps the variant in the subset of variants.

Having identified supporting nucleotide reads for the subject's subsequent sample, in one or more embodiments, the MRD system can generate one or more model parameters for an MRD model that indicates the presence of the tumor within the subject. For example, the MRD system can generate a parameter or hyperparameter for a tumor fraction or variant allele frequency (VAF) in a subsequent sample that corresponds with the tumor fraction or VAF indicated by the tumor profile. In some embodiments, the one or more parameters includes sequencing noise associated with the nucleotide reads (e.g., sequencing data).

In some cases, a subset of variants in a tumor profile—and an MRD model—can be specific to a type of variant, such as an SNV, CNV, or SV. For instance, the MRD system can (i) determine biological observables in a subsequent sample by counting supporting nucleotide reads for the subsequent sample exhibiting target SNVs at genomic coordinates from a tumor profile and (ii) determine, utilizing an SNV-specific MRD model and its corresponding model parameters, a likelihood that the subject comprises residual tumor material based on the count of supporting nucleotide reads exhibiting the target SNVs. As a further example, the MRD system can also (i) determine biological observables by counting nucleotide reads for the subsequent sample that map to copy number variation (CNV) segments within a genome (e.g., non-overlapping bins across a reference genome) and (ii) determine, utilizing a CNV-specific MRD model and its corresponding one or more model parameters, a likelihood that the subject comprises residual tumor material based on the counts of supporting nucleotide reads mapping to the CNV segments.

In addition, or in the alternative to using variant-type-specific MRD models, in some cases, the MRD system can combine likelihoods from multiple variant-type-specific MRD models to determine posterior probabilities that a subject's subsequent sample comprises residual tumor material from the tumor fingerprinted by the tumor profile. For instance, the MRD system can determine a sum of log-likelihoods (or other likelihood combination) based on two or more of a first likelihood that a subject's subsequent sample comprises residual tumor material, as generated by an SNV-specific MRD model; a second likelihood that the subject's subsequent sample comprises residual tumor material, as generated by a CNV-specific MRD model; or a third likelihood that the subsequent sample comprises residual tumor material, as generated by an SV-specific MRD model.

The disclosed MRD system provides several technical advantages over existing systems. For instance, the disclosed MRD system decreases the DNA input for a sample and increases turnaround times for MRD detection relative to existing MRD assay systems. Unlike existing MRD assay systems that rely on bespoke-based probes and require relatively higher plasma volume or cfDNA mass from a sample to determine or evaluate a tumor-specific profile in MRD detection, the disclosed MRD system can leverage less DNA (e.g., less cfDNA) from a sample to generate a tumor profile for a subject and/or determine a likelihood of the presence of residual tumor material in the subject. In some embodiments, for instance, the disclosed MRD system can leverage whole genome sequencing (WGS) or whole exome sequencing (WES) that require relatively less DNA from a sample to sequence sufficient nucleotide reads and determine which supporting nucleotide reads of a subject's sample exhibit variants from a tumor profile. Based on such supporting nucleotide reads from a relatively DNA-lite process and parameters of an MRD model, the disclosed MRD system can likewise determine a likelihood that the subsequent sample comprises residual tumor material. Indeed, the relatively smaller genetic material required for input in creating a tumor profile for an initial sample and/or for sequencing nucleotide reads from a subsequent sample-increases the number of eligible samples for MRD testing relative to existing MRD assay systems. By further leveraging the speed of WGS or WES, in some embodiments, the disclosed MRD system can rely on a high-throughput sequencing device to identify supporting nucleotide reads more quickly from a sample that exhibit variants from a tumor profile relative to existing MRD assay systems. For example, the disclosed MRD system can directly utilize the supporting nucleotide reads generated by WGS or WES without going through the lengthy and resource heavy design, manufacture, and validation processes utilized by existing MRD assay systems.

Beyond reduced DNA input and expedited MRD detection, the MRD system provides more flexibility compared to existing systems. As indicated above, many existing MRD assay systems are limited to panels that are specific to one tumor only for MRD detection and cannot be used for other tumor types or adjusted for variants unique to or unusual for a sample. In contrast to such a one-panel-fits-all-patients approach, the MRD system can not only work across various types of tumors and cancers but also adjust for tumor mutational burdens (TMBs). By identifying a tumor profile comprising a subset of variants specific to a subject's initial sample and within target genomic regions—and further determining, for the subject's subsequent sample, supporting nucleotide reads that exhibit one or more variants of the subset of variants from the tumor profile—the disclosed MRD system can utilize an MRD model and corresponding parameters to generate a likelihood that the subsequent sample includes residual tumor material from different tumor types and based on variants in the supporting nucleotide reads unique or unusual to the subject. Because the disclosed MRD system can identify and utilize a panel that scales with a subject's TMBs, the disclosed MRD system can accurately identify and account for new variants associated with the subject and cancer.

In addition to improved flexibility, the disclosed MRD system provides improved accuracy of MRD detection when compared to existing MRD assay systems. In particular, the MRD system more accurately detects residual tumor material within a sample by better distinguishing between (i) nucleotide reads (e.g., noisy reads) that resemble (but do not constitute) circulating-tumor DNA (ctDNA) and (ii) nucleotide reads that come from or exhibit actual ctDNA (e.g., true positive variants). For example, the MRD system can identify and remove false positive variants arising from noisy reads in the sequencing data that might falsely indicate the presence of ctDNA in a subsequent sample of a subject. To help distinguish such noisy reads, in some embodiments, the MRD system can utilize an MRD model to determine a likelihood that the nucleotide reads from the subsequent sample originate from a variant in the tumor profile versus a likelihood that the nucleotide reads arise from background noise. In some such cases, the disclosed MRD system determines, for each frequency of a range of sample-wide tumor allele frequencies, (i) a first likelihood of a presence of a tumor (e.g., a clean read pileup) given a sample-wide tumor allele frequency and (ii) a second likelihood of an absence of the tumor (e.g., a noisy read pileup) given the sample-wide tumor allele frequency. By better distinguishing noisy reads from tumor-variant-exhibiting reads, the disclosed MRD system improves sensitivity and the accuracy of detecting MRD in a subject relative to existing MRD assay systems.

In addition or in the alternative to the foregoing technical advantages, in some embodiments, the disclosed MRD system performs or facilitates one or more anti-cancer treatments for a subject for which the MRD system has determined that a sample of the subject likely comprises residual tumor material. For example, in some cases, the MRD system (a) compares variants of a tumor profile generated from a subject's initial sample (e.g., FFPE tissue sample) with variants detected in nucleotide reads of the subject's subsequent sample (e.g., plasma sample, liquid biopsy) and (b) utilizes an MRD model to determine a likelihood that the subject's subsequent sample comprises residual tumor material based on the comparison of variants. After determining the subject's subsequent sample comprises such material, the MRD system can identify an anti-cancer therapy for—and/or administer such an anti-cancer therapy to—the subject. As described further below, such an anti-cancer therapy can include, but is not limited to, surgical removal of the cancer, chemotherapy, radiation, antibody therapies, and/or anti-cancer drugs.

As illustrated by the foregoing discussion, the present disclosure utilizes a variety of terms to describe features and advantages of the MRD system. As used herein, the term “sample” refers to a specimen, culture, or the like that is suspected of including a target nucleic acid. In some embodiments, the sample comprises DNA, ribonucleic acid (RNA), peptide nucleic acid (PNA), locked nucleic acid (LNA), chimeric or hybrid forms of nucleic acids as targets. The sample can likewise include any biological, clinical, surgical, agricultural-atmospheric, or aquatic-based specimen containing one or more nucleic acids. A sample also includes any isolated or extracted nucleic acid sample from an organism, such a genomic DNA, fresh-frozen, or formalin-fixed paraffin-embedded nucleic acid specimen. In some cases, accordingly, a sample can include a full genome or partial genome that is isolated or extracted (e.g., in whole or in part by a kit) from an organism and that is prepared to undergo sequencing or an assay in a sequencing device. A sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material, such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The sample can include high molecular weight material, such as genomic DNA (gDNA). The sample can include low molecular weight material such as nucleic acid molecules obtained from formalin-fixed paraffin-embedded (FFPE) or archived DNA samples. In another implementation, low molecular weight material includes enzymatically or mechanically fragmented DNA. The sample can include cell-free circulating DNA. In some implementations, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some implementations, the sample can be an epidemiological, agricultural, forensic, or pathogenic sample. In some implementations, the sample can include nucleic acid molecules obtained from an animal such as a human or mammalian source. In another implementation, the sample can include nucleic acid molecules obtained from a non-mammalian source such as a plant, bacteria, virus, or fungus. In some implementations, the source of the nucleic acid molecules may be an archived or extinct sample or species.

Relatedly, as used herein, the term “initial sample” refers to a sample that is isolated, extracted, or otherwise obtained before a subsequent sample. In particular, an initial sample includes a sample of tissue or bodily fluid taken from a tumor or cancer of a subject. In some embodiments, the initial sample can include cancerous cells and normal cells of the subject. For example, an initial sample from a liquid tumor can include a blood sample from the subject with cancerous cells and normal cells. In embodiments where the tumor is a solid tumor, as a further example, an initial sample can be a small amount of tissue removed from the tumor and/or cancer. In one or more embodiments, an initial sample is taken after a subject is diagnosed with cancer.

As used herein, the term “subsequent sample” refers to a sample that is isolated, extracted, or otherwise obtained after an initial sample. In particular, in certain embodiments, a subsequent sample includes a sample of bodily fluid or marrow of a subject that includes cfDNA. For example, a subsequent sample can include, but is not limited to, bile, blood, bone marrow, plasma, cerebrospinal fluid, saliva, sweat, and urine. In some embodiments, a subsequent sample is taken after or during treatment of a tumor or cancer.

As used herein, the term “subject” refers to an organism from which a sample is derived. In particular, a subject includes an organism from which a sample is isolated or extracted. In some cases, a subject can include a complex organism that has multiple organ systems with different and/or distinct functions. For example, a subject can be a mammal, such as a human, primate, or mouse. In some embodiments, a subject can be a reptile, amphibian, invertebrate, bird, or fish. In one or more implementations, the subject has been diagnosed with a malignancy (e.g., presence of cancer cells or tumor material in the body). Relatedly, the malignancy of the subject may be in remission or active. For example, after receiving treatment for lung cancer, a subject can no longer have cancerous cells in their body.

As further used herein, the term “cancer” refers to uncontrolled growth of abnormal cells in a part of a body. In one or more embodiments, growth of a tumor(s) leads to cancer. As used herein, the term “tumor” refers to an abnormal mass of tissue or abnormal reproduction of cells that forms when cells grow and divide without dying. In some embodiments, a tumor can be benign, precancerous, or malignant. For example, benign tumors are non-cancerous and can be harmless to the body of a subject. Alternatively, tumors can be malignant and cancerous. In some cases, tumors can be solid or liquid. For instance, a solid tumor is a mass of solid cancer cells that grow in organs, bones, muscles, and/or connective tissues. In some cases, solid tumors are categorized based on their originating location in the body and cell type making up the tumor. For example, cell types of solid tumors include, but are not limited to, carcinomas, sarcomas, lymphomas, and carcinosarcomas. In addition to solid tumors being benign or malignant, a liquid tumor can either include benign cells or malignant cells that circulate through the bloodstream, bone marrow, and/or lymphatic system of the subject. Liquid tumors include, but are not limited to, leukemia, lymphoma, and myeloma. In one or more instances, the cells of the tumor can include DNA and fragments of DNA that enter the bodily fluid (e.g., bloodstream, urine, saliva, bone marrow, etc.) of the subject.

As used herein, the term “circulating tumor deoxyribonucleic acid” (or more simply ctDNA) refers to fragments of DNA shed by cancerous or tumor cells that enter the bodily fluid of the subject. For example, ctDNA can include tumor cells circulating in the bone marrow of the subject. Relatedly, as used herein, the term “residual tumor material” refers to ctDNA circulating within a subject related to the tumor and/or cancer found within the subject and associated with a tumor profile.

Relatedly, as used herein, the term “cell free deoxyribonucleic acid” (or more simply cfDNA) refers to DNA shed or otherwise not encapsulated by cells that freely circulates in a bodily fluid, such as blood. In some embodiments, cfDNA can comprise ctDNA in a plasma sample. While cfDNA can include ctDNA, cfDNA can also include DNA that is not tumor-derived.

As used herein, the term “tumor profile” refers to a set of variants or genes that are associated with (e.g., linked to) the presence of a particular tumor or cancer in a subject. In particular, a tumor profile includes a set of variants that are uniquely associated with the presence of the particular tumor in a particular subject. Accordingly, a tumor profile can include specific mutations, variants, and/or biomarkers associated with a tumor of a subject as determined from sequenced DNA from a tumor sample and/or normal sample from the subject. For example, a tumor profile for a solid tumor, such as breast cancer, can have different variants at certain genomic regions and/or genomic coordinates than a tumor profile for a liquid tumor, such as leukemia. In some embodiments, a tumor profile can be specific to a subject, thus a tumor profile for colon cancer from a first subject can differ from a tumor profile for colon cancer from a second subject. In one or more embodiments, the subset of variants from a tumor profile are located at particular genomic regions of the subject. In this disclosure, note that the term “tumor profile” can be interchangeable with the terms “tumor fingerprint” and/or “tumor signature.”

As used herein, the term “generic tumor profile” refers to variants or genes that are characteristic of a particular tumor or cancer. For example, a generic tumor profile may include variants at specific genomic regions that are specific to a tumor or cancer and commonly found among subjects with the tumor or diagnosed with the cancer. To illustrate, the generic tumor profile for pancreatic cancer can include, small number variants, structural variants, and/or copy number variants at specific genomic regions that are associated with pancreatic cancer.

As further used herein, the term “genomic coordinate” (or sometimes simply “coordinate”) refers to a particular location or position of a nucleobase within a genome (e.g., an organism's genome or a reference genome). In some cases, a genomic coordinate includes an identifier for a particular chromosome of a genome and an identifier for a position of a nucleobase within the particular chromosome. For instance, a genomic coordinate or coordinates may include a number, name, or other identifier for a somatic or sex chromosome (e.g., chr1 or chrX) and a particular position or positions, such as numbered positions following the identifier for a chromosome (e.g., chr1:1234570 or chr1:1234570-1234870). In some cases, a genomic coordinate refers to a genomic coordinate on a sex chromosome (e.g., chrX or chrY). Further, in certain implementations, a genomic coordinate refers to a source of a reference genome (e.g., mt for a mitochondrial DNA reference genome or SARS-CoV-2 for a reference genome for the SARS-CoV-2 virus) and a position of a nucleobase within the source for the reference genome (e.g., mt:16568 or SARS-CoV-2:29001). By contrast, in certain cases, a genomic coordinate refers to a position of a nucleobase within a reference genome without reference to a chromosome or source (e.g., 29727).

As used herein, the term “genomic region” refers to a range of genomic coordinates. Like genomic coordinates, in certain implementations, a genomic region may be identified by an identifier for a chromosome and particular positions, such as numbered positions following the identifier for a chromosome (e.g., chr1:1234570-1234870). In various implementations, a genomic region includes positions within a reference genome. In some cases, a genomic region is specific to a particular reference genome. Relatedly, as used herein, the term “target genomic region” refers to a genomic region that is selected or identified for a tumor profile or detection. In particular, a target genomic region can include a range of genomic coordinates associated with a tumor profile for a cancer and/or tumor of a subject. For example, a target genomic region can include (i) a genomic region with well-known associations with a particular cancer and/or tumor or (ii) a genomic region with known associations with a particular cancer and/or tumor of a subject.

As used herein, the term “targeted assay” refers to a sequencing assay of specific genomic regions with known relevance to a specific cancer, tumor, or other disease or diagnostic purpose. For example, a targeted assay can identify variants by sequencing genomic regions with mutations recurrently observed among patients (e.g., tumor hotspots).

As used herein, the term “whole genome sequencing” refers to determining the order of all nucleotides of DNA of the entire genome (or substantially the entire genome) of a subject. In one or more embodiments, whole genome sequencing entails extracting DNA from a sample, shearing the extracted DNA, preparing a DNA or whole genome library, sequencing the whole genome library with a sequencing device, assembling the nucleotide reads, and determining variant or reference calls for the sample based on the nucleotide reads. In some embodiments, whole genome sequencing can translate all DNA base pairs of the subject's genome into a file (e.g., FASTQ, BAM, VCF) comprising letters representing each nucleotide.

As used herein, the term “variant” refers to a nucleobase or multiple nucleobases that do not align with, differs from, or varies from a corresponding nucleobase (or nucleobases) in a reference sequence or a reference genome. For example, a variant can include a single nucleotide variant (SNV), Copy Number Variation (CNV), an insertion and deletion (indel), or a structural variant (SV) that indicates one or more nucleobases in a sample nucleotide sequence differ from nucleobases in corresponding genomic coordinates of a reference sequence or a reference genome. In some embodiments, a variant can be inherited (e.g., germline) or form over a subject's lifetime (e.g., somatic). In certain cases, a somatic variant can occur spontaneously or form in response to stress or damage. In one or more embodiments, a somatic variant can cause cancer or a tumor.

Moreover, as used herein, the term “single nucleotide variant” (SNV) refers to a variation (e.g., substitution) of a single nucleotide at a specific position (e.g., genomic coordinate). For example, at a particular location, an SNV involves replacing adenine with a guanine at a specific genomic coordinate. In some embodiments, an SNV can be a single nucleotide polymorphism (SNP), an SNV that is present at a certain frequency in a population of subjects.

As used herein, the term “copy number variant” (CNVs) refers to a variation in the number of copies of a specific segment of DNA at a position (e.g., genomic coordinate or genomic region). In some cases, a CNV can include insertions, deletions, or duplications of segments of DNA. In some embodiments, a CNV can be at least 50 base pairs.

Further, as used herein, the term “structural variant” refers to a variation (e.g., deletion, insertion, translocation, inversion) in a structure of an organism's chromosome or a variation to nucleotide sequences of the organism's chromosome (e.g., a sample genomic sequence). In some cases, a structural variant includes a variation to a threshold number of base pairs (e.g., >50 base pairs) within an organism's chromosome. Accordingly, in certain implementations, a structural variant includes an insertion or deletion exceeding a threshold number of base pairs, a duplication exceeding a threshold number of base pairs, an inversion, a translocation, or a copy number variation (CNV). While some examples of structural variants use 50 base pairs as a threshold number of base pairs, in some embodiments, the threshold number of base pairs for a structural variant may be different, such as 16, 25, 32, 35, 45, 100, or 1,000 base pairs.

Additionally, as used herein, the term “panel of normals” refers to a set of samples that establish a baseline. In particular, a panel of normals can include a set of normal samples that establish a baseline for MRD detection. To illustrate, a panel of normals can include a set of samples from various non-cancer subjects. In some implementations, the MRD detection system uses a panel of normals as data generated from the panel of normals should not be indicative of MRD (e.g., does not indicate the presence of ctDNA).

Also, as used herein, the term “nucleotide read” (or simply “read”) refers to an inferred or predicted sequence of one or more nucleobases (or nucleobase pairs) from all or part of a sample genomic sequence (e.g., a sample genomic sequence, complementary DNA). In particular, a nucleotide read includes a determined or predicted sequence of nucleobase calls for a nucleotide fragment (or group of monoclonal nucleotide fragments) from a sequencing library corresponding to a sample. For example, in some embodiments, the MRD system determines a nucleotide read by generating nucleobase calls for nucleobases passed through a nanopore of a nucleotide-sample slide, determined via fluorescent tagging, or determined from a well in a flow cell. In some cases, a nucleotide read can refer to a particular type of read, such as a nucleotide read synthesized from sample library fragments that are shorter than a threshold number of nucleobases (e.g., SBS reads). In these or other cases, another type of nucleotide read can refer to (i) assembled nucleotide reads that have been assembled from shorter nucleotide reads to form a contiguous sequence (e.g., assembled nucleotide reads) satisfying a threshold number of nucleobases, (ii) circular consensus sequencing (CCS) reads satisfying the threshold number of nucleobases, or (iii) nanopore long reads satisfying the threshold number of nucleobases.

As used herein, the term “supporting nucleotide reads” refers to nucleotide reads that include or otherwise indicate the presence of a variant or gene from a tumor profile. In particular, supporting nucleotide reads refers to a nucleotide read (e.g., a paired-end read) that includes or has been identified as including one or more variants from a subset of variants within a tumor profile. For instance, in certain implementations, a supporting nucleotide read includes a paired-end read (i) corresponding to (e.g., generated from) an initial sample of a subject (e.g., a tumor sample) and (ii) having a first read mate and/or a second read mate that overlap with a genomic region associated with a tumor profile and support a tumor allele or variant associated with the tumor sample. To illustrate, supporting nucleotide reads for an SNV at a specific genomic coordinate for the tumor profile could include nucleotide reads from the subsequent sample that include the same SNV (e.g., alternative allele) at the target genomic region. In some embodiments, the supporting nucleotide reads include germline variants and noise from the sequencing data.

As further used herein, the term “nucleobase call” (or simply “base call”) refers to a determination or prediction of a particular nucleobase (or nucleobase pair) for an oligonucleotide (e.g., nucleotide read) during a sequencing cycle or for a genomic coordinate of a genomic sample. In particular, a nucleobase call can indicate a determination or prediction of the type of nucleobase that has been incorporated within an oligonucleotide on a nucleotide-sample slide (e.g., read-based nucleobase calls). In some cases, for a nucleotide read, a nucleobase call includes a determination or a prediction of a nucleobase based on intensity values resulting from fluorescent-tagged nucleotides added to an oligonucleotide of a nucleotide-sample slide (e.g., in a cluster of a flow cell). As suggested above, a single nucleobase call can be an adenine (A) call, a cytosine (C) call, a guanine (G) call, a thymine (T) call, or an uracil (U) call. Note that the terms nucleobase and nucleotide base are interchangeable.

As used herein, the term “allele” refers to one of two or more possible nucleotide bases and/or nucleotide sequences that occur at a given genomic coordinate or genomic region. Generally, a human individual inherits two alleles, one from each parent. In some cases, the allele presents as a variant or alternative form of a gene. In some cases, alleles can be SNPs, insertions, and/or deletions.

As used herein, the term “allele frequency” refers to a measurement (e.g., percentage or ratio) indicating a frequency or prevalence of an allele within a given population. In some embodiments, the MRD system determines the allele frequency by dividing the number of occurrences of the allele at a genomic coordinate in the given population by the total number of alleles at the genomic coordinate across the given population. For instance, in some embodiments, the MRD system can determine the allele frequency for a variant in the subset of variants. In particular, if the variant corresponds to an alternative allele, the MRD system can identify the number of occurrences for the alternative allele associated with the variant and divide the number of occurrences by the total number of alleles at the genomic coordinate of the variant.

As used herein, the term “minimal residual disease model” (or simply MRD model) refers to a framework with one or more parameters that facilitate determining a likelihood that observed data (e.g., sequencing data, set of nucleotide reads, etc.) is compatible with the tumor profile. More specifically, the MRD model can determine a likelihood that the supporting nucleotide reads indicate the presence of residual tumor material (e.g., ctDNA) in a sample. In some embodiments, the MRD model is a mathematical or statistical model. For example, the MRD model can be, but is not limited to a probability model, linear model, continuous model, discrete model, or a differential equation.

As used herein, the term “parameter” refers to a quantity, value, or variable for a model. In some cases, the parameter can be estimated from data (e.g., sequencing data). For example, a parameter can represent a value of noise associated with a supporting nucleotide read. In one or more instances, the parameters are based on the type of variant in the subset of variants. For example, a parameter can be the length and/or content of the supporting nucleotide reads. In some cases, a parameter can reflect knowledge of the binding affinities of the targeted assay. In one or more embodiments, the one or more parameters can include hyperparameters. Accordingly, such a hyperparameter need not be estimated from internal data (e.g., sequencing data) but from an external source.

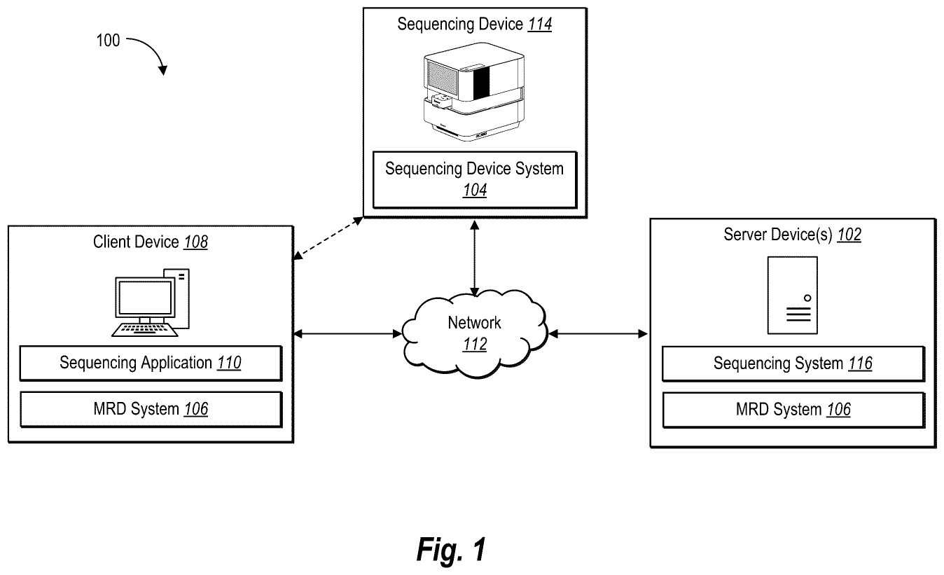

The following paragraphs describe the MRD system with respect to illustrative figures that portray example embodiments and implementations. FIG. 1 illustrates a schematic diagram of a computing system 100 in which a minimal residual disease (MRD) system 106 operates in accordance with one or more embodiments. As illustrated, the computing system 100 includes one or more server device(s) 102 connected to a client device 108 and a sequencing device 114 via a network 112. While FIG. 1 shows one embodiment of the MRD system 106, this disclosure describes alternative embodiments and configurations below.

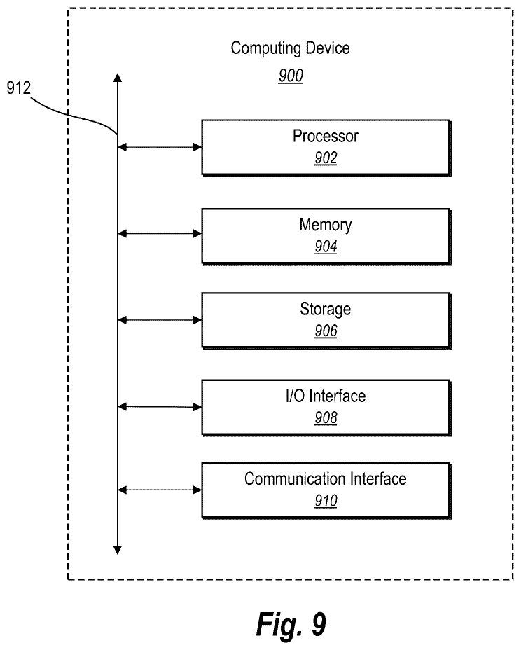

As shown in FIG. 1, the server device(s) 102, the client device 108, and the sequencing device 114 are connected via the network 112. Accordingly, each of the components of the computing system 100 can communicate via the network 112. The network 112 comprises any suitable network over which computing devices can communicate. Example networks are discussed in additional detail below with respect to FIG. 9.

As indicated by FIG. 1, the sequencing device 114 comprises a computing device and a sequencing device system 104 for sequencing a sample or other nucleic-acid polymer. In some embodiments, by executing the sequencing device system 104 using a processor, the sequencing device 114 analyzes nucleotide fragments or oligonucleotides extracted from samples to generate nucleotide reads or other data utilizing computer-implemented methods and systems either directly or indirectly on the sequencing device 114. More particularly, the sequencing device 114 receives nucleotide-sample slides (e.g., flow cells) comprising nucleotide fragments (e.g., genomic DNA fragments) extracted from samples and further copies and determines the nucleobase sequence of such extracted nucleotide fragments in the form of nucleotide reads.

In one or more embodiments, the sequencing device 114 utilizes SBS to sequence nucleotide fragments into nucleotide reads and determine nucleobase calls for the nucleotide reads. In addition, or in the alternative to communicating across the network 112, in some embodiments, the sequencing device 114 bypasses the network 112 and communicates directly with the server device(s) 102 or the client device 108. By executing the sequencing device system 104, the sequencing device 114 can further store the nucleobase calls as part of base-call data that is formatted as a binary base call (BCL) file and/or fast-all quality (FASTQ) file and send the BCL file and/or FASTQ file to the client device 108 and/or the server device(s) 102.

As further indicated by FIG. 1, the server device(s) 102 may generate, receive, analyze, store, and transmit digital data, such as data for amino-acid sequences or nucleotide sequences. As shown in FIG. 1, the sequencing device 114 may send (and the server device(s) 102 may receive) various data from the sequencing device 114, including data representing amino-acid sequences or nucleotide sequences (e.g., base calls and/or corresponding base-call-quality scores). The server device(s) 102 may also communicate with the client device 108. In particular, the server device(s) 102 can send data representing amino-acid sequences or nucleotide sequences (e.g., base calls and/or corresponding base-call-quality scores) to the client device 108.

Additionally, as shown in FIG. 1, the server device(s) 102 can include the MRD system 106. In one or more embodiments, as explained further below, the MRD system 106 analyzes nucleotide reads generated from various samples-including an initial sample and/or a subsequent sample—to determine a tumor profile and/or detect residual tumor material as part of MRD. To illustrate, the MRD system 106 can use an MRD model determine a likelihood that the subsequent samples include residual tumor material based on their corresponding (e.g., supporting) nucleotide reads. In some embodiments, the MRD system 106 can use an additional MRD model to determine an additional likelihood that the subsequent sample comprises residual tumor material. Further the MRD system 106 can combine the likelihood that the subsequent sample comprises residual tumor material with the additional likelihood that the subsequent sample comprises the residual tumor material to generate a posterior probability that subsequent sample comprises residual tumor material.

In some embodiments, the server device(s) 102 comprise a distributed collection of servers where the server device(s) 102 include a number of server devices distributed across the network 112 and located in the same or different physical locations. Further, the server device(s) 102 can comprise a content server, an application server, a communication server, a web-hosting server, or another type of server.

As further shown in FIG. 1, the server device(s) 102 can include a sequencing system 116. Generally, the sequencing device system 104 analyzes sequencing data received from the sequencing device 114 to determine nucleotide sequences for whole genomic samples or other nucleic-acid polymers. For example, the sequencing system 116 can receive raw data (e.g., base-call data for nucleotide reads) from the sequencing device 114 and determine a nucleic acid sequence for a sample. To illustrate, the sequencing system 116 can receive data for nucleotide reads from the sequencing device 114, align the nucleotide reads with a reference sequence or reference genome, and generate genotype calls (e.g., variant or reference calls) for a sample based on the aligned nucleotide reads and sequencing metrics (e.g., quality scores). In some embodiments, the sequencing system 116 determines the sequences of nucleobases from a sample including DNA and/or RNA.

In some cases, the server device(s) 102 is located at or near a same physical location of the sequencing device 114 or remotely from the sequencing device 114. Indeed, in some embodiments, the server device(s) 102 and the sequencing device 114 are integrated into a same computing device. The server device(s) 102 may run software on the sequencing device 114 or the MRD system 106 to generate, receive, analyze, store, and transmit digital data, such as by sending or receiving tumor profiles, nucleotide reads and/or MRD detection results. In some embodiments, the sequencing device 114 or the MRD system 106 store and access a database or table of likelihoods of residual tumor material for subsequent samples and/or MRD detection results.

As further illustrated and indicated in FIG. 1, the client device 108 can generate, store, receive, and send digital data. In particular, the client device 108 can receive data for amino-acid sequences or nucleotide sequences (or variants thereof), scores generated for various samples, or MRD detection results determined for a subsequent sample from the server device(s) 102 and/or the sequencing device 114. The client device 108 can accordingly present data concerning the amino-acid sequences or nucleotide sequences (or variants thereof), the likelihood of residual tumor material generated for the subsequent samples, or the MRD detection results determined with respect to the sample within a graphical user interface to a user associated with the client device 108.

The client device 108 illustrated in FIG. 1 may comprise one of various types of client devices. For example, in some embodiments, the client device 108 includes a non-mobile device, such as a desktop computer or server, or another type of client device. In yet other embodiments, the client device 108 includes a mobile device, such as a laptop, a tablet, a mobile telephone, or a smartphone. Additional details with regard to the client device 108 are discussed below with respect to FIG. 9.

As further illustrated in FIG. 1, the client device 108 include a sequencing application 110. The sequencing application 110 may be a web application or a native application on the client device 108 (e.g., a mobile application, desktop application, etc.). The sequencing application 110 can include instructions that (when executed) cause the client device 108 to receive or request data from the MRD system 106 and present data from the sequencing device 114. Furthermore, the sequencing application 110 can comprise instructions that (when executed) cause the client device 108 to provide a graphical visualization of the likelihood that the subsequent sample includes residual tumor material.

As illustrated in FIG. 1, the MRD system 106 may be located on the client device 108 or on the server device(s) 102. Accordingly, in some cases, the MRD system 106 is implemented by (e.g., located entirely or in part) on the client device 108. As mentioned, in yet other cases, the MRD system 106 is implemented by one or more other components of the computing system 100, such as the server device(s) 102. In particular, the MRD system 106 can be implemented in a variety of different ways across the server device(s) 102, the network 112, the client device 108, and/or the sequencing device 114. Although FIG. 1 illustrates the MRD system 106 as housed or hosted by the client device 108 and the server device(s) 102, in some embodiments, the client device 108 houses or hosts some or all components of the MRD system 106. Further, in some embodiments, the server device(s) 102 house or host some or all components of the MRD system 106. Accordingly, the MRD system 106 can be housed or hosted by one or more of the client device 108 or the server device(s) 102.

Though FIG. 1 illustrates the components of the computing system 100 communicating via the network 112, in some cases, the components of the computing system 100 can also communicate directly with each other, bypassing the network 112. For instance, and as previously mentioned, in some implementations, the client device 108 communicates directly with the sequencing device 114. Additionally, in some embodiments, the client device 108 communicates directly with the MRD system 106. Moreover, the MRD system 106 can access one or more databases housed on or accessed by the server device(s) 102 or elsewhere in the computing system 100.

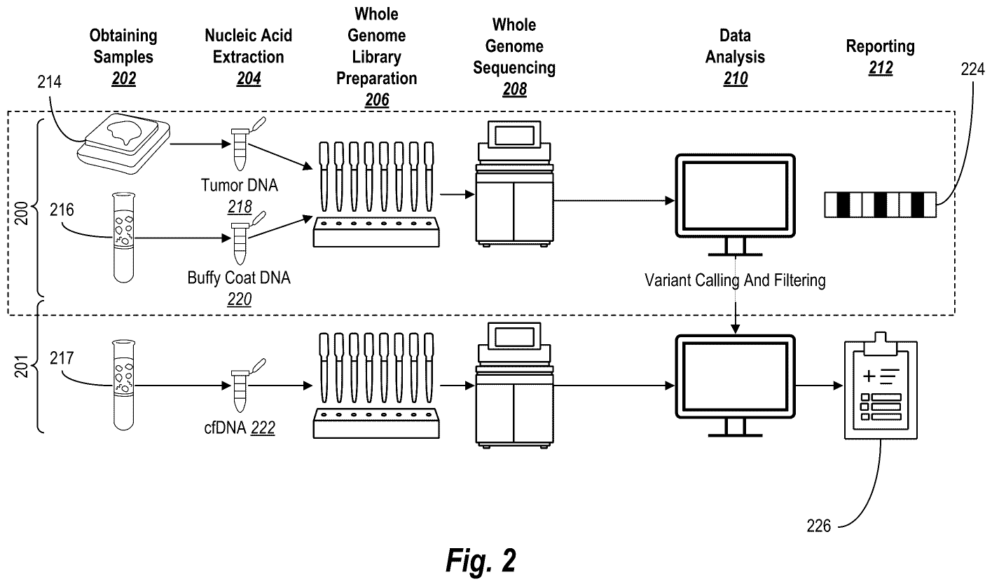

As discussed above, in some cases, the MRD system 106 can analyze an initial sample and a subsequent sample from a subject diagnosed with cancer or a tumor to determine if the subsequent sample has residual tumor material. In accordance with one or more implementations, FIG. 2 illustrates a tumor-profile pipeline 200 of the MRD system 106 for processing initial sample(s) of a subject to generate a tumor profile 224 and a recurrent-test pipeline 201 of the MRD system 106 for processing a subsequent sample of the subject to determine a likelihood of whether the subsequent sample comprises residual tumor material as part of a detection result 226.

As illustrated in both the tumor-profile pipeline 200 and the recurrent-test pipeline 201 of the MRD system 106 in FIG. 2, the MRD system 106 performs operations (or uses components from prior operations by clinicians or other systems) for obtaining samples 202, nucleic acid extraction 204, whole genome library preparation 206, whole genome sequencing 208, data analysis 210, and reporting 212. In the tumor-profile pipeline 200, as shown in FIG. 2, the MRD system 106 optionally performs such operations to generate the tumor profile 224. After the tumor-profile pipeline 200 has been completed, as further shown in FIG. 2, the MRD system 106 performs such operations in the recurrent-test pipeline 201 to determine whether subsequent sample(s) 217 comprises residual tumor material as part of the detection result 226. While the MRD system 106 performs operations (or uses components from prior operations by clinicians or other systems) for the tumor-profile pipeline 200 before the recurrent-test pipeline 201, for conciseness, the following paragraphs sometimes describe operations in each pipeline together with respect to obtaining samples 202, nucleic acid extraction 204, whole genome library preparation 206, whole genome sequencing 208, data analysis 210, and reporting 212.

As shown in FIG. 2, for example, the MRD system 106 can perform MRD detection using various types of samples. For instance, the MRD system 106 can perform MRD detection using one or more initial samples. As depicted, the initial sample(s) 214 can include a sample obtained from a tumor (e.g., via a biopsy of the tumor) of a subject diagnosed with a particular type of cancer (e.g., breast, liver, blood). In some cases, the initial sample(s) 214 can include a formalin-fixed paraffin-embedded (FFPE) sample. In one or more embodiments, the initial sample(s) 214 can comprise a fresh frozen sample for which a biopsy of the tumor was snap-frozen in liquid nitrogen within 30-60 minutes of excision.

As further shown in FIG. 2 as part of the tumor-profile pipeline 200, the MRD system 106 can access or generate the tumor profile 224 using one or more normal samples, such as normal sample(s) 216. In particular, the normal sample(s) 216 can be obtained via a blood draw from a subject. In some instances, the MRD system 106 uses one or more components of the normal sample(s) 216 (e.g., obtained via centrifuging the normal sample(s) 216) to identify variants specific to the subject that are not related to the tumor. Indeed, the MRD system 106 can use one or more of a plasma sample, a buffy coat sample, or a sample of red blood cells from other normal sample(s) 216, to aid in somatic variant filtering.

As FIG. 2 illustrates as part of the recurrent-test pipeline 201, the MRD system 106 can further perform MRD detection using one or more subsequent samples. As depicted, the subsequent sample(s) 217 can be a sample taken after the initial sample and/or treatment of the tumor. In one or more embodiments, the subsequent sample(s) 217 can be obtained via a blood draw from the subject and the MRD system 106. In certain implementations, the MRD system 106 can use one or more of a plasma sample, a buffy coat sample, or a sample of red blood cells (e.g., obtained via centrifuging the subsequent sample(s) 217). In some embodiments, the subsequent sample(s) 217 comes from other bodily fluids with cfDNA. For example, in one or more implementations, the subsequent sample(s) 217 can be a bone marrow sample, a urine sample, a saliva sample, a stool sample, or a bile sample comprising cfDNA.

As further illustrated in FIG. 2, the MRD system 106 can use DNA extracted from the initial sample(s) 214, the normal sample(s) 216 in the tumor-profile pipeline 200 and the DNA extracted from the subsequent sample(s) 217 in the recurrent-test pipeline 201, respectively, to prepare for DNA sequencing on a flow cell or other nucleotide-sample slide. For example, the MRD system 106 can use tumor DNA 218 extracted from the initial sample(s) 214 and/or the buffy coat DNA 220 extracted from the normal sample(s) 216 that is prepared for processing in library templates to be seeded in a nucleotide-sample slide. Further, the MRD system 106 can use cfDNA 222 extracted from the subsequent sample(s) 217 that is prepared for processing in library templates to be seeded in a nucleotide-sample slide.

Additionally, as illustrated in FIG. 2, the MRD system 106 (or an associated sequencing device) can receive or use nucleotide-sample slides (e.g., flow cells) comprising oligonucleotide clusters for which whole genome library preparation 206 has been performed on the samples. As part of the tumor-profile pipeline 200, in some embodiments, the MRD system 106 optionally receives or uses oligonucleotide clusters on a nucleotide-sample slide that have been prepared from a library by fragmenting the tumor DNA 218 extracted from the initial sample(s) 214, and the buffy coat DNA 220 extracted from the normal sample(s) 216. As part of the recurrent-test pipeline 201, in some cases, the MRD system 106 similarly receives or uses oligonucleotide clusters on a nucleotide-sample slide that have been prepared from a library by fragmenting the cfDNA 222 extracted from the subsequent sample(s) 217. In some cases, the MRD system 106 (or an associated sequencing device) uses library templates within which adaptors have been ligated to (i) fragments of the tumor DNA 218 or the buffy coat DNA 220 as part of the tumor-profile pipeline 200 or (ii) fragments of the cfDNA 222 as part of the recurrent-test pipeline 201. Additionally, in one or more embodiments, the MRD system 106 can amplify the DNA fragments with the ligated adaptors with a polymerase chain reaction (PCR). Subsequently, in some implementations, the MRD system 106 can pool the DNA fragments with the ligated adaptors into a library template.

As further shown in FIG. 2, the MRD system 106 can perform whole genome sequencing 208 using the sequencing device 114 (e.g., NovaSeq 6000, NovaSeq X, or other sequencing device) as part of both the tumor-profile pipeline 200 and the recurrent-test pipeline 201. In particular, the MRD system 106 can use whole genome sequencing 208 to generate (a) nucleotide reads for the initial sample(s) 214 and/or the normal sample(s) 216 in the tumor-profile pipeline 200 and (b) nucleotide reads for the subsequent sample(s) 217 in the recurrent-test pipeline 201. In some embodiments, the MRD system 106 generates such nucleotide reads by generating paired-end reads. Indeed, the MRD system 106 can generate a plurality of paired-end reads for each of the initial sample(s) 214, normal sample(s) 216, and the subsequent sample(s) 217.

While FIG. 2 depicts whole genome library preparation 206 and whole genome sequencing 208, in some embodiments, the MRD system 106 can use whole exome sequencing (WES) and/or tumor hotspot panels. Accordingly, in both the tumor-profile pipeline 200 and the recurrent-test pipeline 201, the MRD system 106 or an associated sequencing device can (i) receive or use nucleotide-sample slides comprising oligonucleotide clusters for which whole exome library preparation has been performed on the initial sample(s) 214, the normal sample(s) 216, and/or the subsequent sample(s) 217 and (ii) perform WES using such nucleotide-sample slides on a sequencing device (e.g., the sequencing device 114).

After performing WGS, WES, or tumor hotspot panels to generate nucleotide reads for the initial sample(s) 214 and/or normal sample(s) 216 in the tumor-profile pipeline 200, as shown in FIG. 2, the MRD system 106 can analyze nucleotide reads from the initial sample(s) 214 and/or normal sample(s) (216) as part of the data analysis 210. As indicated by the data analysis 210 in FIG. 2, in some embodiments, the MRD system 106 determines variant calls or reference calls for the initial sample(s) 214 and/or the normal sample(s) 216 by comparing the nucleotide reads of the initial sample(s) 214 and/or the normal sample(s) 216 to a reference genome. Some or all of the variant calls at target genomic regions become part of the tumor profile 224. For instance, as shown in FIG. 2, during the data analysis 210, the MRD system 106 perform variant filtering. In particular, the MRD system 106 compares variants determined from the initial sample(s) 214 and the normal sample(s) 216. Based on variants determined from the initial sample(s) 214 matching variants in the normal sample(s) 216, the MRD system 106 can identify variants (germline or clonal hematopoiesis mutations of indeterminate potential) within the subject that do not arise from the tumor and exclude those variants from the tumor profile 224. As indicated above, the tumor profile 224 can include variants or genes indicating (or associated with) a presence of a tumor or cancer in a subject from which the initial sample(s) 214 have been isolated or extracted.

While FIG. 2 depicts the MRD system 106 performing the tumor-profile pipeline 200 to generate the tumor profile 224, the MRD system 106 need not perform the tumor-profile pipeline 200 but merely access or otherwise identify the tumor profile 224 that has been previously generated (e.g., by a different system or by the same system). Accordingly, the tumor-profile pipeline 200 for generating the tumor profile 224 is optional for the MRD system 106. To indicate that the tumor-profile pipeline 200 is optional, FIG. 2 encompasses the tumor-profile pipeline 200 with a dotted line.

As FIG. 2 further illustrates, the MRD system 106 can likewise analyze nucleotide reads of the subsequent sample(s) 217 as part of the data analysis 210 in the recurrent-test pipeline 201. In the data analysis 210, the MRD system 106 determines variant or reference calls for the subsequent sample(s) 217 with respect to a reference genome. As explained in more detail below, the MRD system 106 can access or otherwise identify the tumor profile 224 for the initial sample(s) 214 and use an MRD model and one or more model parameters to analyze supporting nucleotide reads from the nucleotide reads of the subsequent sample(s) 217.

As further shown in FIG. 2, based on the data analysis 210 in the recurrent-test pipeline 201, the MRD system 106 can generate the detection result 226. In particular, the MRD system 106 determines whether or not the subsequent sample(s) 217 has residual tumor material. In one or more embodiments, the MRD system 106 determines a likelihood that the subsequent sample(s) 217 includes residual tumor material. In some cases, the MRD system 106 provides the detection result 226 (e.g., an indication of MRD or residual tumor material) for display within a graphical user interface of a client device (e.g., the client device 108).

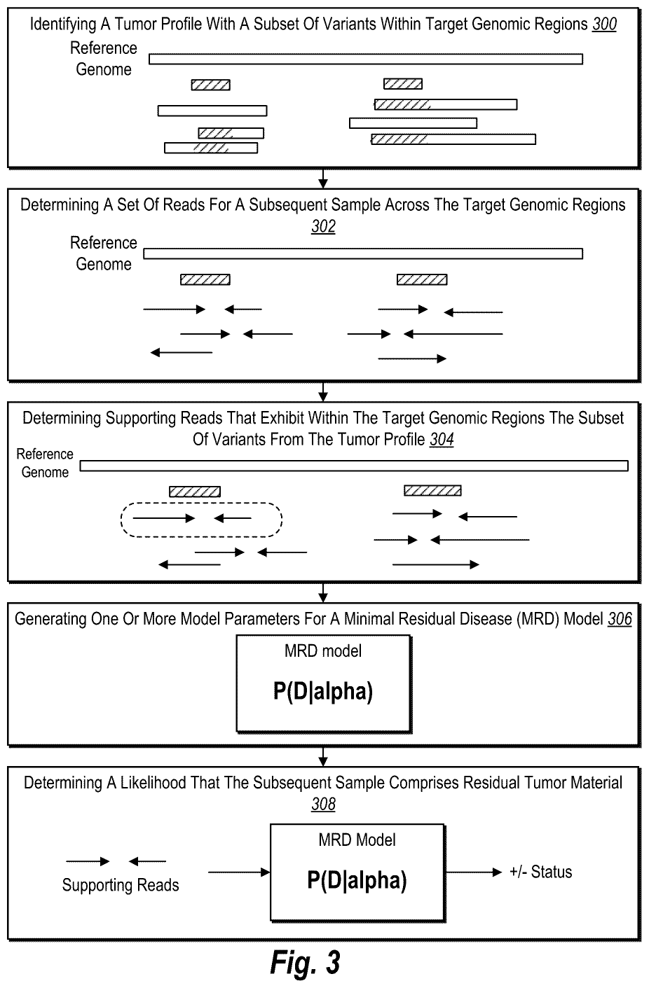

As described above, the MRD system 106 can perform MRD detection using an initial sample of a tumor and/or cancer and/or normal sample from the subject to identify the tumor profile for the subject followed by a subsequent sample of a subject to detect residual or recurrent tumor material. In accordance with one or more implementations, FIG. 3 illustrates an overview of the MRD system 106 identifying a tumor profile from an initial sample of a subject comprising a tumor and determine a likelihood that a subsequent sample of the subject comprises residual tumor material based on nucleotide reads for the subsequent sample.

As shown in FIG. 3, the MRD system 106 can perform the act 300 of identifying a tumor profile with a subset of variants within target genomic regions. For example, the MRD system 106 can identify target genomic regions having variants that are uniquely associated with the presence of the tumor and/or cancer within the subject. To determine or identify which variants are part of a tumor profile, as shown in FIG. 3, the MRD system 106 can compare one or more nucleotide reads generated form the initial sample to a reference genome. In some embodiments, the MRD system 106 can align the one or more nucleotide reads for an initial tumor sample of a subject with the reference genome and compare the aligned one or more nucleotide reads to the reference genome to identify variants differing from the reference bases. The MRD system 106 can likewise and separately align one or more nucleotide reads for a normal sample of the subject with the reference genome and compare the aligned one or more nucleotide reads to the reference genome to identify variants differing from the reference bases. Based on a comparison of variants from the tumor sample and variants from the normal sample of the subject, the MRD system 106 can determine which variants within target genomic regions are relevant to a tumor and, accordingly, part of a tumor profile. Thus, the MRD system 106 can determine a subset of variants from the one or more nucleotide reads, establishing the tumor profile at target genomic regions. Because generating such a tumor profile is optional as indicated above, however, the MRD system 106 can also merely access or otherwise identify a tumor profile that has been previously generated based on an initial tumor sample and/or initial normal sample of a subject.

By accessing or establishing the tumor profile for the subject, in some embodiments, the MRD system 106 accesses or establishes a baseline for what the presence of cancer and/or the tumor looks like in the subject. In particular, the MRD system 106 identifies or establishes a link between the particular type of cancer and/or tumor and the subject diagnosed with cancer and/or tumor on a nucleic-acid level (e.g., establishes the nucleic-acid indicators). Further, the MRD system 106 identifies the location of variants at target genomic regions within the genome of the subject with respect to the reference genome. Thus, as will be described further below, the MRD system 106 can use the tumor profile and the target genomic regions associated with the tumor profile to guide the residual tumor material detection process.

As further shown in FIG. 3, after identifying a tumor profile for a subject, the MRD system 106 can perform an act 302 of determining a set of reads for a subsequent sample across the target genomic regions. As indicated above, the MRD system 106 can compare one or more nucleotide reads from the subsequent sample with a reference genome. For instance, the MRD system 106 can align the nucleotide reads of the subsequent sample with the reference genome and compare the aligned nucleotide reads to the reference genome to identify variants differing from the reference bases. Based on the comparison of aligned reads with the reference genome, the MRD system 106 can determine variant calls for the subsequent sample within target genomic regions relevant to the tumor profile. For example, the MRD system 106 can determine if the subsequent sample includes SNVs (e.g., SNPs), CNVs, SVs, indels, etc. within and/or outside of the target genomic regions.

From among the nucleotide reads of the subsequent sample, as further illustrated in FIG. 3, the MRD system 106 can perform the act 304 of determining supporting reads that exhibit, within the target genomic regions, one or more variants of the subset of variants from the tumor profile. In particular, the MRD system 106 can determine if variants at target genomic regions in the subsequent sample match and/or are mapped to the subset of variants in the tumor profile within the target genomic regions, as described above.

After identifying or determining such supporting nucleotide reads, as FIG. 3 further illustrates, the MRD system 106 can perform the act 306 of generating one or more model parameters for an MRD model. Indeed, the MRD system 106 can generate one or model parameters that indicate a presence of a tumor within the subject. As discussed in more detail below, in some cases, the MRD system 106 determines the one or more parameters of the MRD model based on a type of variant (e.g., SNV, CNV, SV, etc.) in the subset of variants of the tumor profile. For example, based on the subset of variants comprising SNVs, the MRD system 106 can utilize the estimated variant allele frequency and noise of the sequenced data as parameters for the MRD model.

By utilizing the MRD model and its parameters, as further shown in FIG. 3, the MRD system 106 can perform an act 308 of determining a likelihood that the subsequent sample comprises residual tumor material. For example, the MRD system 106 can utilize the MRD model and one or more parameters to analyze the supporting nucleotide reads. As set forth below, in some embodiments, the MRD system 106 can utilize the MRD model to determine a likelihood that the supporting nucleotide reads originate from noise or from variants associated with the tumor and/or cancer.

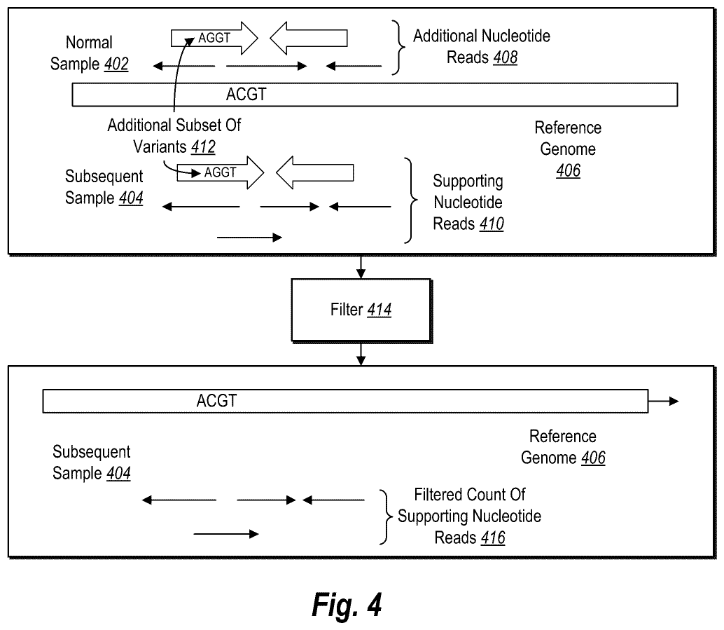

As previously mentioned, in one or more embodiments, the MRD system 106 performs personalized MRD detection with respect to a tumor profile determined for a subject. Relatedly, the MRD system 106 identifies a subset of variants for the tumor profile from the initial sample and looks for the subset of variants in the subsequent sample. In some embodiments, the MRD system 106 avoids calling false positive variants in the subset of variants by filtering the nucleotide reads of the initial sample and subsequent sample. FIG. 4 illustrates the MRD system 106 filtering supporting nucleotide reads in accordance with one or more implementations.

As mentioned above, in some embodiments, the MRD system 106 identifies a subset of variants at target genomic regions in the initial sample from a tumor or cancer. By contrast, certain genomic regions include variants that are not associated with the tumor and/or cancer, including some target genomic regions. For example, during a lifetime, somatic cells (cells not part of the germline) can change or mutate resulting in somatic variants that are non-cancerous. In one or more implementations, somatic variants do not occur in every cell in the body of the subject and are not inherited. Indeed, in some cases, the target genomic regions can include somatic variants unrelated to the cancer or tumor. Moreover, a subject can include germline (also called constitutional) variants or mutations that occur in a gamete or gamete producing cells. Such mutations are found in every cell (or nearly every cell) of the body and can be passed down from one generation to another (e.g., from a mother or father to a child). Like somatic variants, germline variants can be non-cancerous.

As shown in FIG. 4, the MRD system 106 can identify and filter out somatic and/or germline variants unrelated to the tumor and/or cancer. For example, as shown in FIG. 4, the MRD system 106 can receive a normal sample 402. As used herein, the term “normal sample” refers to a sample of bodily tissue of the subject that does not include tumor and/or cancer cells or material. In one or more implementations, the normal sample comes from a biological organ or system of the subject affected by cancer. For example, a normal sample from a subject diagnosed with lung cancer can come from non-cancerous or non-tumor lung tissue.

FIG. 4 shows an implementation where the MRD system 106 can generate additional nucleotide reads 408 for the normal sample 402 by performing WGS or WES on the normal sample 402. As described above, the MRD system 106 can align the additional nucleotide reads 408 of the normal sample 402 to a reference genome 406 and identify an additional subset of variants 412. As indicated above, the additional subset of variants 412 can include somatic, clonal hematopoiesis mutations of indeterminate potential (CHIP), and/or germline variants that are not associated with the tumor and/or cancer of the initial sample from the subject.

As further shown in FIG. 4, the MRD system 106 can identify the additional subset of variants 412 in supporting nucleotide reads 410 of a subsequent sample 404. As FIG. 4 illustrates, the MRD system 106 can apply a filter 414 that removes or excludes the additional subset of variants 412 from the supporting nucleotide reads 410 of the subsequent sample 404. In one or more embodiments, the filter 414 comprises a software program that identifies genomic regions where the additional subset of variants 412 matches the variants from the supporting nucleotide reads 410 of the subsequent sample 404 and excludes those supporting nucleotide reads 410 from further analysis. Indeed, in one or more implementations, the MRD system 106 can determine a filtered count of supporting nucleotide reads 416 for the subsequent sample 404.

Additionally, in one or more embodiments, the filter 414 can remove supporting nucleotide reads in difficult-to-call or difficult-to-align genomic regions or coordinates. Such difficult-to-call or difficult-to-align genomic regions may include genomic regions that historically (or for a given sample) include nucleotide reads that frequently fail to align well with a linear reference genome or produce variant or reference calls that exhibit low-quality base-call-quality scores and mapping quality scores below normal thresholds. For example, in some cases, the MRD system 106 can utilize the filter 414 to remove genomic regions, genomic coordinates, and/or non-overlapping bins that are known to be noisy, difficult to align, and/or hard to call. In some embodiments, the MRD system 106 applies the filter to the initial sample to generate a more accurate tumor profile.

In addition or in the alternative to a single filter for the filter 414, in one or more embodiments, the MRD system 106 can apply a chain of filters to each supporting nucleotide reads 410 exhibiting a target SNV. For example, the MRD system 106 can apply a read filter, nucleotide filter, location filter, and additional MRD model filters to ensure the quality and accuracy of the additional MRD model.

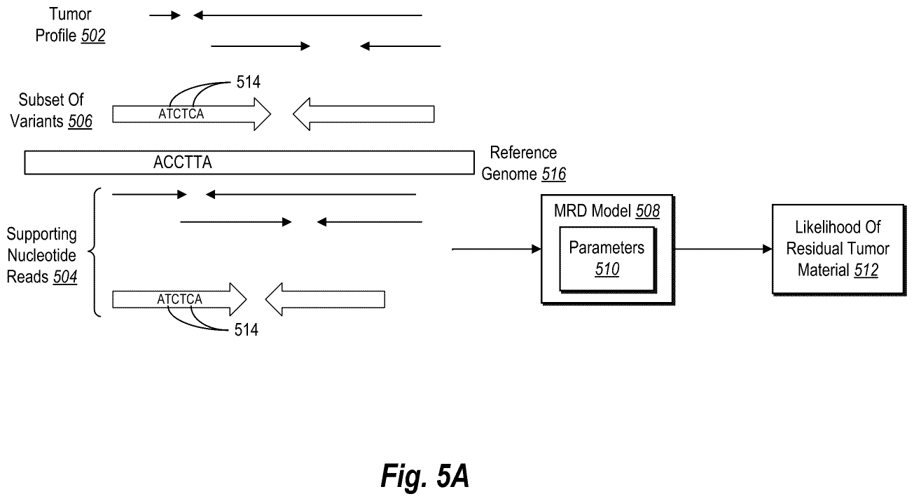

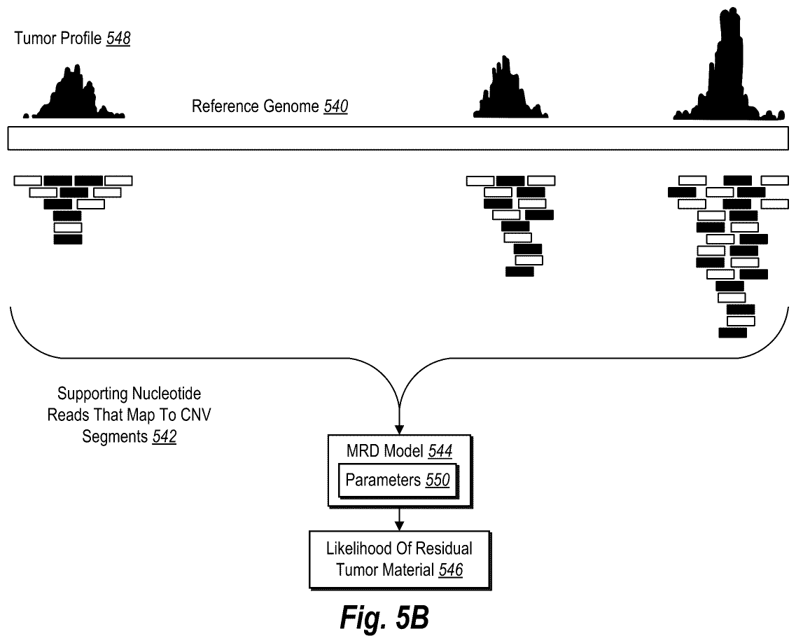

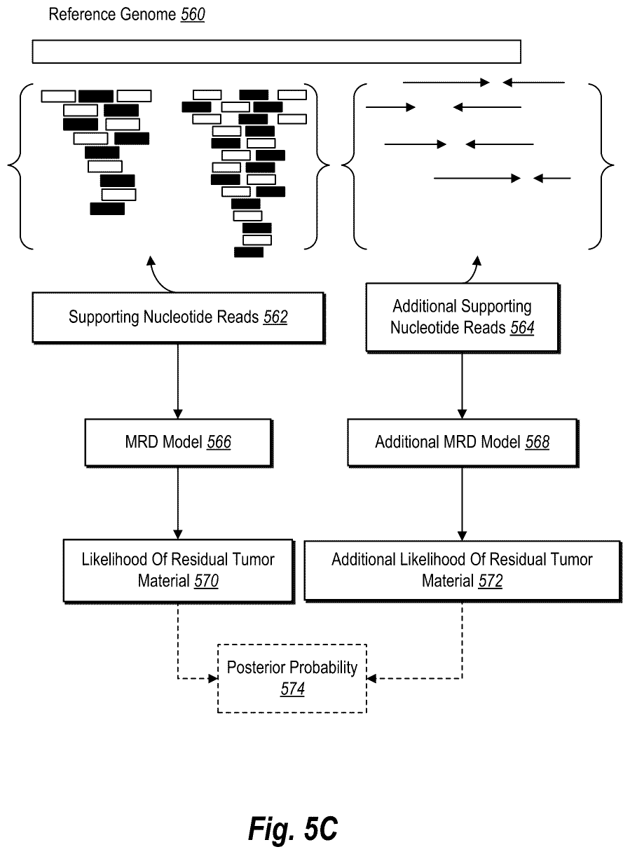

As previously mentioned, the MRD system 106 uses supporting nucleotide reads exhibiting variants from a tumor profile for an initial sample of a subject and an MRD model to determine a likelihood of residual tumor material in a subsequent sample. In accordance with one or more implementations, FIGS. 5A-5C illustrate the MRD system 106 utilizing an MRD model to determine a likelihood that a subsequent sample of a subject comprises residual tumor material based on such supporting nucleotide reads. As an overview, FIG. 5A shows the MRD system 106 utilizing an MRD model to determine a residual-tumor-material likelihood based on supporting nucleotide reads that exhibit target single nucleotide variants (SNVs) from the tumor profile. FIG. 5B illustrates the MRD system 106 determining residual-tumor-material likelihood based on supporting nucleotide reads mapping to copy number variant (CNV) segments. FIG. 5C illustrates the MRD system 106 determining a residual-tumor-material likelihood based on supporting nucleotide reads the exhibit different types of variants (e.g., SNVs, CNVs, SVs). The following paragraphs describe each figure in turn.

As illustrated in FIG. 5A, for example, the MRD system 106 can identify a tumor profile 502 with a subset of variants 506 within target genomic regions that are associated with the tumor and/or cancer specific to the subject. Moreover, the MRD system 106 can determine a count of supporting nucleotide reads 504 that exhibit the subset of variants 506 from the tumor profile 502. As described further below, in one or more embodiments, the MRD system 106 determines the count of supporting nucleotide reads 504 based on the type of variant in the subset of variants. In some embodiments, the supporting nucleotide reads 504 can include germline variants or noise that is present in the tumor profile 502. In alternative embodiments, the MRD system 106 can utilize a generic tumor profile with a subset of variant that related to the specific tumor and/or cancer.

As shown in FIG. 5A, in one or more embodiments, the MRD system 106 can generate for the MRD model 508 one or more parameters 510 for the MRD model 508 to determine if the supporting nucleotide reads 504 are compatible with the tumor profile 502. For example, the MRD system 106 can generate the one or more parameters 510 of the MRD model 508 based on the (i) noise associated with target genomic regions or genomic coordinates, (ii) tumor fraction in a subsequent sample, (iii) variant allele frequency of the subsequent sample, (iv) ploidy of the initial sample, and/or (v) other data associated with the subsequent sample or population genetics at target genomic regions of the tumor profile 502. To illustrate, the MRD system 106 can generate a noise parameter and/or tumor fraction parameter that indicate the presence of the tumor and/or cancer in the subject. In some embodiments, the MRD system 106 can determine the parameters based on the type of variant exhibited by the subset of variants 506 from the tumor profile 502. For example, the MRD system 106 can generate one or more parameters for a subset of variants exhibiting SNVs that differ from the one or more parameters for a subset of variants exhibiting CNVs from the tumor profile 502. Moreover, one or more parameters for the subset of variants exhibiting SVs can differ from the one or more parameters for a subset of variants exhibiting indels.

In one or more embodiments, the MRD system 106 include optional parameters and or models that include prior knowledge of a targeted assay. For example, the MRD system 106 can include parameters reflecting the targeted assay by including knowledge about panel of normals, the binding affinities, biases, chemical make-up, sample preparation, etc. to generate parameters that can be utilized by the MRD model 508 or that can adjust the supporting nucleotide reads 504 so that such target-assay parameters account for prior knowledge of the targeted assay.

As further illustrated in FIG. 5A, the MRD system 106 can utilize the one or more parameters 510 with the MRD model 508 to determine a likelihood of residual tumor material 512 in the subsequent sample based on the supporting nucleotide reads 504. For example, in some embodiments, the MRD model 508 employs a probability model (e.g., Bayesian estimator, frequentist approach, Poisson distribution, binomial distribution, etc.) with the one or more parameters 510 on the supporting nucleotide reads 504. To illustrate, in one or more embodiments, the MRD system 106 can utilize a noise parameter and tumor fraction parameter with the MRD model 508 to scan a parameter space (e.g., hyper-parameter space) to select a max-log-likelihood ratio of the tumor fraction given the noise at target genomic regions based on the supporting nucleotide reads 504 from the subsequent sample. In some cases, the MRD system 106 can scan the hyper-parameter space using grid search, gradient descent, etc. In some implementations, the MRD system 106 can utilize the max-log-likelihood of the tumor fraction and generate a posterior probability indicating the likelihood of residual tumor material 512 in the subsequent sample. As described in more detail below, in some cases, the MRD system 106 can generate a max-log-likelihood ratio for individual MRD models associated with a type of variant (e.g., SNV, CNV, SV, etc.) and combine the log-likelihood ratios from individual MRD models.

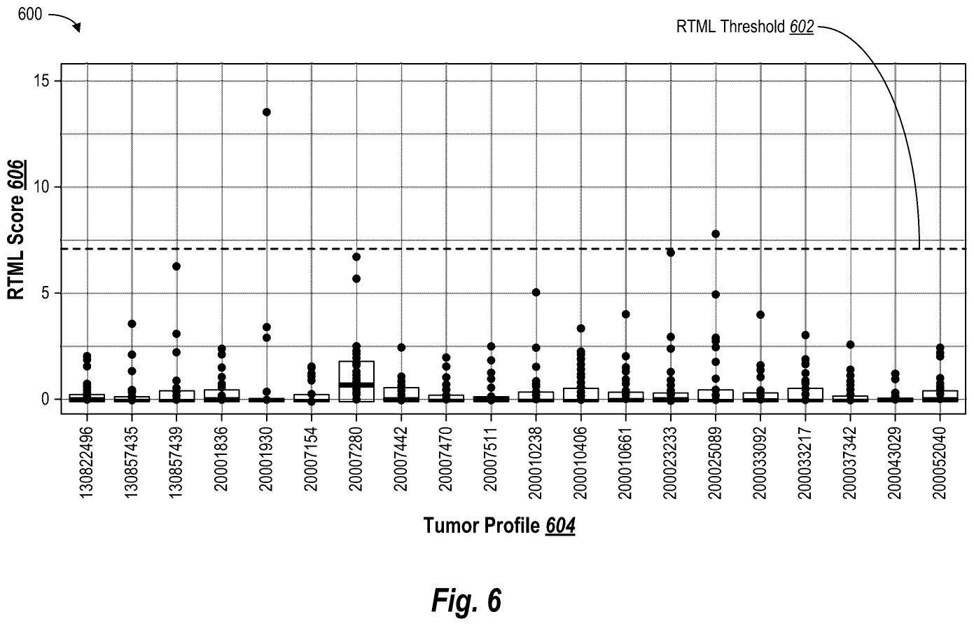

Additionally, in one or more embodiments, the MRD system 106 can determine the presence of residual tumor material within a subject. For example, in some cases, the MRD system 106 can determine a residual tumor material likelihood threshold. This disclosure describes and depicts examples of such a residual tumor material likelihood threshold below with respect to FIG. 6. In some implementations, based on the likelihood that the subsequent sample includes residual tumor material exceeding the residual tumor material likelihood threshold, the MRD system 106 can determine the presence of the residual tumor material within the subject.

As just indicated, in some cases, the MRD system 106 can determine the parameters for the MRD model 508 based on the type of variants within the subset of variants 506. For example, in certain implementations, the one or more parameters associated with a subset of variants exhibiting SNVs can differ from the one or more parameters associated with a subset of variants exhibiting CNVs.

As shown in FIG. 5A, the MRD system 106 can identify the tumor profile 502 with the subset of variants 506 exhibiting target SNVs 514 at genomic coordinates corresponding to variants from the tumor profile 502. In some embodiments, the tumor profile 502 with the subset of variants 506 exhibiting the target SNVs 514 can reflect molecule counts and/or variant counts associated with the tumor and/or cancer. The MRD system 106 can determine a count of supporting nucleotide reads 504 that exhibit the target SNVs 514 at low frequency (e.g., SNV with an allele frequency less than 1% in the DNA of a subject). For example, from the set of nucleotide reads of the subsequent sample, the MRD system 106 can identify and/or count the supporting nucleotide reads 504 that exhibit and/or match the target SNVs 514 at genomic coordinates from the tumor profile 502. If the tumor profile 502 includes 3,476 of the target SNVs 514 at 1,000; 2,500; or 3,476 genomic coordinates, for example, the MRD system 106 can determine a number of the supporting nucleotide reads 504 exhibiting the target SNVs 514 at the same 1,000; 2,500; or 3,476 genomic coordinates, respectively, from the tumor profile 502.

In some cases, the MRD system 106 can generate the one or more parameters 510 indicating a presence of the tumor and/or cancer in the subject. Indeed, in one or more embodiments, the MRD system 106 can generate the one or more parameters 510 that filter out the target SNVs 514 at genomic coordinates that are not compatible with the tumor profile 502. As indicated above, the MRD system 106 system can determine if the supporting nucleotide reads 504 are compatible with the tumor profile 502 by determining if the supporting nucleotide reads 504 exhibit the target SNVs 514 from the tumor profile 502 or variants caused by sequencing errors and, therefore, represent noise. Indeed, the MRD system 106 can estimate the noise for a given sample (e.g., initial sample or subsequent sample) and remove the supporting nucleotide reads 504 that exhibit variants representing noise.

In one or more cases, the MRD system 106 can filter out noise by generating a pseudo tumor profile (or pseudo fingerprint). As used herein, the term “pseudo tumor profile” refers to a generated set of genes, variants, or proteins that closely mirror the genes, variants, and/or proteins associated with (e.g., linked to) a tumor profile of a subject. For example, a pseudo tumor profile can include the subset of variants 506 (e.g., target SNVs 514) in the tumor profile 502. In one or more embodiments, the pseudo tumor profile can include signal probes and noise probes that establish a floor of the noise for the subsequent sample.

Relatedly, a signal probe can include a template of a nucleotide sequence at a genomic coordinate that mirrors or resembles a nucleotide sequence of a target SNV in a tumor profile. In one or more embodiments, the MRD system 106 can generate a signal probe for each supporting nucleotide reads 504 exhibiting a target SNV that meets one or more quality metrics (e.g., QSCORE, MAPQ, etc.).

By contrast, a noise probe can include a template of a nucleotide sequence at genomic coordinates unrelated to a cancer and/or tumor in a tumor profile. In some embodiments, the MRD system 106 generates noise probes within a defined range from signal probes. For example, in one or more embodiments, the MRD system 106 can place noise probes within 3, 5, 10, 15, or 20 kilobases, or another threshold number of kilobase pairs on either side of the signal probes. Additionally, in some cases, the noise probes have similar genomic context as the nearby signal probe. For example, the noise probes can have the same nucleotides, 3-nucleotide counting transition and/or 5-nucleotide counting transition as the signal probe.

In some implementations, the MRD system 106 utilizes the signal probes and noise probes to determine whether the amplitude of a signal from the subsequent sample arises from noise in the sequencing data. As indicated above, the MRD system 106 can identify true positive SNVs (e.g., target SNVs 514) that appear at low frequencies. For example, based in part on the coverage of the subsequent sample, the MRD system 106 can determine, utilizing the MRD model 508, if the supporting nucleotide reads 504 exhibit target SNVs 514.

As shown in FIG. 5A, in one or more embodiments, the MRD system 106 can determine the supporting nucleotide reads 504 by counting the number of DNA molecules and/or fragments from the supporting nucleotide reads 504 that overlap a reference nucleotide and/or variant nucleotide at each signal probe and noise probe. In some cases, the MRD system 106 can utilize the count of supporting nucleotide reads 504 and the one or more parameters and the MRD model to determine if the signal probes and noise probes are compatible with the estimated concentration of the tumor, estimated variant allele frequency (VAF) of the tumor, and/or estimated tumor fraction.

As just mentioned, the MRD system 106 can estimate the noise and VAF of the subsequent sample by utilizing the noise probes and signal probes. In one or more implementations, the MRD system 106 estimates the VAF by subtracting the estimated noise from the combined amplitude of signal and noise. For example, the estimated VAF can be modeled as: α̅=α̅+̅∈̅−∈̅,̅ where (̅α̅) represents the estimated VAF, (α̅+̅∈̅) represents the combined amplitude of the signal and noise, and (∈̅)̅ represents the estimated noise.

In certain implementations, the MRD system 106 estimates the noise at genomic coordinates by taking a sample-based approach with the noise probes. For example, the MRD system 106 can model the noise for the noise probes as: v̅=dN(α+∈)≈Σᵣ₌₁^{N}^{_{filtered}}δ^{snv}(r); where (̅v̅)̅ represents the expected observed count of target SNVs, (α) represents the variant allele frequency, (∈) represents the noise, (N_{filtered}) represents the supporting nucleotide reads 504 exhibiting target SNVs 514, and (δ^{snv}(r)) represents a detector function where:

$${δ}^{snv} ( r ) = \{ \begin{matrix}1 & read r exhibits SNV \\ 0 & otherwise\end{matrix}$$

In alternative embodiments, the MRD system 106 can estimate the noise by running the aforementioned equation on a panel of normals or linear regression.

In one or more embodiments, after the MRD system 106 estimates the noise in the sequencing data, the MRD system 106 can remove nucleotide reads from the supporting nucleotide reads 504 exhibiting target SNVs 514 at genomic coordinate where the observable number of the target SNVs 514 significantly exceeds the expected value for the subsequent sample with the estimated VAF (a). As indicated above, in some cases, the coverage of the subsequent sample can affect the expected observable number of the target SNVs 514. For example, a subsequent sample with a higher coverage may have a higher expected observable number of the target SNVs 514. In this example, coverage includes an average number of nucleotide reads that align to and/or cover a known genomic coordinate and/or region of a reference genome 516. To illustrate, a sample for which a sequencing device sequences each base across genomic regions of the sample's genome on average 30 times has a coverage of 30×. In some cases, a higher coverage indicates more confidence in the accuracy of the sequencing data and variant calling because the sequencing device sequences a larger number of DNA molecules and/or fragments. In some cases, 1% of the reads at a genomic coordinate are expected to be invalid. Thus, in an embodiment with 10 samples at a coverage of 100× each, 10 reads at the genomic coordinates may be invalid.

In one or more embodiments, the MRD system 106 can determine whether the supporting nucleotide reads 504 exhibiting the target SNVs 514 of the subsequent sample exceeds the expected value for the estimated VAF (α̅) by utilizing the VAF (α), noise, and a Poisson distribution. In particular, given the coverage of the subsequent sample, the MRD system 106 determines the likelihood of two supporting nucleotide reads exhibiting the target SNVs 514 at the same genomic coordinate. In one or more embodiments, the likelihood of observing two supporting nucleotide reads 504 exhibiting target SNV 514 in the observed data can be modeled as:

$$p ( {n}_{observedvariants} \vert 2 α ) \approx \frac{{( 2 {N}_{p} α )}^{n}}{n !} {e}^{- α} < 0.01$$

where p(n_{observedvariants}|2α) represents the likelihood of two supporting nucleotide reads exhibiting the target SNV 514 at the same genomic coordinate, (Nₚ) represents the number of somatic variants at a given position, (α) represents the VAF, and (∈) represents the noise. For example, in an embodiment where the coverage of the supporting nucleotide reads 504 exhibiting the target SNVs 514 is 30×, it is expected for 0.3 supporting nucleotide reads 504 exhibiting target SNVs 514 at each genomic coordinate are invalid. Inserting this value into the equation above indicates that the genomic coordinate of any noise probe or signal probe with more than one or two discordant nucleotide reads should be excluded from further analysis because they are unlikely to be compatible with the tumor profile 502.

As further shown, in FIG. 5A, after the MRD system 106 removes noisy genomic coordinates by removing the incompatible noise probes and signal probes, the MRD system 106 can determine a likelihood of residual tumor material 512 in the subsequent sample. For example, the MRD system 106 can utilize the MRD model 508 and the one or more parameters 510 representing the VAF (α) and noise (∈) in the sequencing data (D) to determine the likelihood of residual tumor material. For example, in one or more embodiments, the MRD model 508 is a Bayes Estimator that generates a likelihood (e.g., prior probability) for a target SNV at a genomic coordinate.

For example, in some embodiments, the MRD system 106 determines the likelihood p(D|alpha) according to the following equation:

$$p ( D \vert alpha ) = {\prod }_{r ∈ D} ( \begin{matrix}( 1 - α - ϵ ( r ) ) & if Wildtype \\ α + ϵ ( r ) & otherwise\end{matrix} )$$

where p(D|alpha) represents the likelihood of the supporting nucleotide reads 504 given the VAF, (α) represents the VAF, and (∈(r)) represents the empirical noise amplitude. In one or more embodiments, the MRD system 106 can determine the empirical noise based on the noise probes, the noise from a panel of normal, or fitting the sequencing data from the noise probes and a panel of normals.

As mentioned above, the MRD system 106 can determine a likelihood of residual tumor material in a subsequent sample for different variant types. In accordance with one or more embodiments, FIG. 5B illustrates the MRD system 106 determining a likelihood that a subsequent sample of the subject comprises residual tumor material based on counts of supporting nucleotide reads mapping to copy number variant (CNV) segments from a tumor profile. In some implementations, the MRD system 106 generates one or more parameters to estimate expected bin coverages corresponding to genomic regions for an entire genome or exome of a subsequent sample. As discussed in more detail below, the MRD system 106 utilizes the expected bin coverages to determine if the supporting nucleotide reads that map to CNV segments 542 arise from noise or a true CNV.

As shown in FIG. 5B and discussed above, in one or more embodiments, the MRD system 106 can identify or generate a tumor profile 548 with a subset of variants. In certain implementations, the MRD system 106 identifies or generates the tumor profile 548 that highlights CNVs by identifying genomic regions (e.g., segments) associated with tumor specific CNVs. For example, the MRD system 106 can align nucleotide reads of a subsequent sample with a reference genome 540 and identify CNVs that correspond with specific tumor and/or cancer of the subject. In one or more embodiments, the tumor profile 548 includes the ploidy of a tumor specific CNV segment and the ploidy for a normal CNV segment.

In one or more embodiments, the MRD system 106 can determine the supporting nucleotide reads by generating pileups of supporting nucleotide reads that map to the CNV segments 542 at target genomic regions. Indeed, the MRD system 106 can further determine a count of supporting nucleotide reads that map to CNV segments 542 found in the tumor profile 548. For example, the MRD system 106 can count the number supporting nucleotide reads that map into non-overlapping bins that correspond to genomic regions. In one or more embodiments, the non-overlapping bins can vary in size. For example, a first bin can span 10 kilobase pairs and a second bin can span 20 kilobase pairs. Similarly, a first bin can span 5 kilobase pairs and a second bin can span 15 kilobase pairs. Further, a bin can comprise different number of bases, such a 5, 10, 15, 20, 25, or 30 kilobase pairs.

In some cases, the MRD system 106 determines the count of supporting nucleotide reads that map to the CNV segments 542 in each non-overlapping bin by combining the size of the non-overlapping bin and the coverage of the subsequent sample. For instance, if the non-overlapping bin spans 10 kilobase pairs with a coverage of 30×, the size of the bin will be 300,000 base pairs. Moreover, in one or more embodiments, the ploidy (e.g., number of set of chromosomes in a cell) of the subject affects the coverage. For example, in genomic regions where ploidy is higher, the coverage increases.

As described below, in some embodiments, the MRD system 106 determines a sample-wide likelihood of residual tumor material by analyzing the CNV segments across all (or some) of the non-overlapping bins spanning the genome. In particular, based on the estimated tumor fraction of the tumor, the MRD system 106 can determine if the count of supporting nucleotide reads that map to the CNV segments 542 exceed or fall below the estimated tumor fraction of the tumor. For example, if the estimated tumor fraction of the tumor profile is 0.01%, the count of the supporting nucleotide reads that map to the CNV segments 542 across all of the non-overlapping bins should be elevated by 0.01%. To further illustrate, if the size of the non-overlapping bin is 300,000 base pairs, the count of supporting nucleotide reads that map to the CNV segments 542 should be elevated by 30. In one or more cases, the MRD system 106 observes this elevation pattern across thousands of bins and utilizes the MRD model 544 and one or more parameters 550 to determine the likelihood of residual tumor material 546.

As shown in FIG. 5B, the MRD system 106 can generate an MRD model 544 with the one or more parameters 550. For example, in one or more embodiments, the one or more parameters 550 can include a first likelihood of the presence of the tumor given a sample-wide tumor allele frequency, which can act as a proxy for an estimated sample-wide tumor fraction (e.g., maximum sample-wide tumor allele frequency), noise, length of supporting nucleotide reads that map to the CNV segments 542, length of CNV segments, ploidy for CNV segments, etc. In certain implementations, the MRD system 106 utilizes the one or more parameters 550 and the MRD model 544 to determine a first likelihood of the presence of the tumor given the sample-wide likelihood that the supporting nucleotide reads that map to CNV segments are compatible with the tumor profile 548 and a second likelihood of the absence of the tumor given the sample-wide likelihood that the supporting nucleotide reads that map to CNV segments are compatible with the tumor profile 548.

To illustrate such likelihoods, in one or more embodiments, the MRD system 106 generates (i) a first likelihood of the presence of the tumor by determining a likelihood that a non-overlapping bin comprises a clean pileup of supporting nucleotide reads that map to CNV segments 542 and (ii) a second likelihood of an absence of the tumor by determining a likelihood of a noisy pile up of supporting nucleotide reads that map to CNV segments 542. In one or more embodiments, for instance, the MRD system 106 calculates, for each range of sample-wide tumor allele frequencies, a (i) clean pileup likelihood P(RIF, clean) and (ii) noisy pileup likelihood P(R|noisy) in each non-overlapping bin. To illustrate, in one or more embodiments, the range of sample-wide tumor allele frequencies can be logarithmic (e.g., 0, 0001, 0.0002, 0.0004 . . . , 0.1204). In some cases, the MRD system 106 can combine the first (e.g., clean pileup) likelihood P(R|F, clean) and second (e.g., noisy pileup) likelihood P(R|noisy) and determine sample-wide likelihoods for each sample-wide tumor allele frequency.

In some embodiments, the MRD system 106 can determine a clean pileup likelihood P(R|F, clean) over a range of position-specific alt allele frequencies. For example, the clean pile up likelihood can be modeled as the following equation:

$$P ( R \vert F , clean ) = {\sum }_{f} \frac{P ( R \vert F , clean )}{{N}_{f}}$$

where P(R|F, clean) represents a clean pileup likelihood given the sample-wide tumor allele frequency (f) represents position-specific alt allele frequencies under consideration, and N_{f} represents the number of frequency values from [0, F]. In one or more embodiments, summing the first likelihoods for individual supporting nucleotide reads that map to the CNV segments 542 considers the fragment length and contents. For example, the MRD system 106 can utilize the tumor specific and normal specific fragment length distributions to combine a range of position-specific alt allele frequencies under consideration (f) from 0 to the sample-wide tumor allele frequency (F). In one or more embodiments, the sample-wide tumor allele frequency is a maximum sample-wide tumor allele frequency.

In addition to determining such likelihoods, in particular embodiments, the MRD system 106 can determine the normalized tumor and normal ploidys for each CNV segment (k). In one or more cases, the MRD system 106 can further determine the probabilities that a tumor and/or normal fragment map to a CNV segment (k) associated with a non-overlapping bin. In one or more embodiments, the probabilities are proportional to the length of the CNV segment multiplied by the ploidy. For instance, in some cases, the MRD system 106 determines the probability that an arbitrary supporting nucleotide read (e.g., cfDNA read) will map to CNV segment (k) for each sample-wide tumor allele frequency (F). Subsequently, the MRD system 106 can determine a sample-wide likelihood for each of the segments according to the following equation:

$$P ( S \vert F ) = C {\prod }_{k} {{p}_{k} ( F )}^{{s}_{k}}$$

where P(S|F) represents the likelihood that the set of the per-segment fragment counts for the subsequent sample have a maximum sample-wide tumor allele frequency, C represents a constant, pₖ represents the ploidy for a CNV, (F) represents a maximum sample-wide tumor allele frequency, and (Sₖ) represents the number of supporting nucleotide reads that map to CNV segments 542 associated with a non-overlapping bin.

Having determined position-specific pileup likelihood for a range of sample-wide tumor allele frequencies, the MRD system 106 can determine a sample-wide likelihood by accumulating the position-specific pileup likelihoods according to the following equation:

$$P ( D \vert F ) = {\prod }_{j} P ( {R}_{j} \vert F ) {\prod }_{k} P ( {S}_{k} \vert F )$$

where P(D|F) represents the likelihood of the subsequent sample having a maximum sample-wide tumor allele frequency (F), P(Rⱼ|F) represents the probability that a nucleotide read pair having the maximum sample-wide tumor allele frequency (F), and P(Sₖ|F) represents a likelihood that the number of supporting nucleotide reads mapped to the CNV segments 542 have the estimated tumor fraction (F). In alternative to a Bayesian estimator as described above, in some embodiments, the MRD model 544 can be a multinomial model, Dirichlet-multinomial model, or gamma-Poisson model.

In one or more cases, the MRD system 106 can determine a likelihood that the estimated tumor fraction is nonzero based on the supporting nucleotide reads that map to CNV segments 542. For example, as discussed below the MRD system 106 can determine the presence of residual tumor material by utilizing a residual tumor likelihood threshold. Based on the likelihood that the subsequent sample includes residual tumor material exceeding the residual tumor material likelihood threshold, the MRD system 106 can determine the presence or absence of the residual tumor material.

As discussed above, in one or more embodiments, the MRD system 106 can combine individual likelihoods from MRD models analyzing different variant types. In accordance with one or more embodiments, FIG. 5C illustrates the MRD system 106 determining a likelihood of residual tumor material based on analyzing supporting nucleotide reads the exhibit different types of variants. For example, as shown in FIG. 5C and as described above, the MRD system 106 can align supporting nucleotide reads 562 and additional supporting nucleotide reads 564 to a reference genome 560. Moreover, in one or more embodiments, the MRD system 106 can determine if the supporting nucleotide reads 562 map to CNV segments indicated in the tumor profile. Likewise, in some cases, the MRD system 106 can determine if the additional supporting nucleotide reads 564 exhibit target SNVs at genomic coordinates in the tumor profile.

As further shown in FIG. 5C, the MRD system 106 can analyze the supporting nucleotide reads 562 with MRD model 566. In certain implementations, the MRD system 106 can analyze the additional supporting nucleotide reads 564 with an additional MRD model 568. In one or more implementations, the one or more parameters of the MRD model 566 differ from the one or more parameters of the additional MRD model 568 because the respective models include parameters for specific types of variants. As described above in reference to FIG. 5B, the MRD system 106 can determine a likelihood of residual tumor material 570 with the MRD model 566 and an additional likelihood of residual tumor material 572 with the additional MRD model 568. In some embodiments, the likelihood of residual tumor material 570 and the additional likelihood of residual tumor material 572 comprise max-loglikelihood ratios. In some cases, the MRD system 106 can independently generate the max-log-likelihood ratio for each model.

As indicated above, the residual-tumor-material likelihoods generated by respective MRD models can still be used to determine the presence or absence of residual tumor material. In one or more embodiments, the MRD system 106 can determine the presence or absence of the tumor within the subject based on the likelihood of residual tumor material 570. For example, based on the likelihood of residual tumor material 570, the MRD system 106 can determine if the tumor has a nonzero tumor fraction. Moreover, the MRD system 106 can further determine the presence or absence of the tumor based on the additional likelihood of residual tumor material 572. But the MRD system 106 can also generate a more accurate determination of the presence or absence of the residual tumor material by analyzing residual-tumor-material likelihoods for more than one variant type.

Based on multiple residual-tumor-material likelihoods, as further shown in FIG. 5C, the MRD system 106 can generate a posterior probability 574 indicating that a subsequent sample includes residual tumor material. For example, the MRD system 106 can individually perform a Bayes operation on the likelihood of residual tumor material 570 and the additional likelihood of residual tumor material 572 and combine the posterior probabilities. Alternatively, the MRD system 106 can combine the max-log-likelihood ratios prior to applying the Bayes operation. In one or more embodiments, the MRD system 106 can determine the presence or absence of the tumor based on the posterior probability 574.

While FIG. 5C shows the MRD system 106 generating a posterior probability for a subset of variants comprising CNVs and SNVs, the MRD system 106 can include any number of MRD models that correspond to different variant types (e.g., SVs, methylation, indels, etc.).

As just discussed, the MRD system 106 can utilize one or more MRD models to generate one or more likelihoods and/or posterior probabilities that a subsequent sample comprises residual tumor material. As indicate above, the MRD system 106 decreases the DNA input for a sample, increases sample eligibility for MRD testing and detection, and decreases turnaround times for MRD detection relative to existing MRD assay systems. As shown below, Table 1 illustrates the success rate of extracting cfDNA from 4 mL of plasma of a 10 mL blood sample at different yield targets. As shown by Table 1, the success rate of extracting 5 ng of cfDNA from 4 mL of plasma in 907 cancer samples was 97.9% and in 186 normal samples was 93.5%. As further indicated by Table 1, the likelihood of successfully extracting cfDNA decreases as the extraction yield increases to 30 ng of cfDNA and further to 60 ng of cfDNA. In particular, a clinician can successfully extract at least 5 ng of cfDNA from a cancer sample 97.9% or from a normal sample 93.5% but can successfully extract at least 60 ng of cfDNA from a cancer sample 25.8% or from a normal sample 2.7%.

TABLE 1

| Extraction | | | | | |
|---|---|---|---|---|---|
| yield from | | | | | |
| 4 ml plasma | >5 ng | >10 ng | >15 ng | >30 ng | >60 ng |
| % of cancer | 97.9% | 87.6% | 74.5% | 48.4% | 25.8% |
| samples | | | | | |
| (n = 907) | | | | | |
| % of normal | 93.5% | 70.4% | 46.2% | 15.1% | 2.7% |
| samples | | | | | |
| (n = 186) | | | | | |

Because the MRD system 106 can us between 2 ng and 5 ng of cfDNA extracted from merely 4 ml of a subject's sample—and determine a tumor profile for an initial sample of a subject and/or determine a likelihood that a subsequent sample comprises residual tumor material—the MRD system 106 exhibits a distinct advantage over existing MRD assay systems. In contrast to the MRD system 106, existing MRD assay systems require more volume of plasma than 4 ml and more mass of cfDNA than 5 ng from a sample to determine either a tumor profile or perform some type of existing MRD. Relative to existing MRD assay systems, therefore, the MRD system 106 decreases the DNA input for a sample to perform MRD while accurately identifying subjects with residual tumor material.

As noted above, the MRD system 106 can determine the presence or absence of the tumor within the subject by determining a residual tumor material likelihood above or below a threshold. In accordance with one or more implementations, FIG. 6 illustrates the MRD system 106 determining the presence of residual tumor material by utilizing a residual tumor material likelihood threshold from among residual tumor material scores. As used herein, the term “residual tumor material likelihood threshold” (or RTML threshold) refers to a threshold, limit, or MRD score that reflects the limit of detection (LOD) indicating the presence of residual tumor material.

To obtain the results in FIG. 6 and determine a threshold for LOD of residual tumor material in a subsequent sample, researchers conducted a sub-study using data shown in Table 2. As shown in Table 2, the MRD system 106 assessed tumor profiles from 20 tumor samples to determine the residual tumor material likelihood. As further indicated in Table 2, the sub-study used tumor samples across a range of cancer types (e.g., breast cancer, melanoma, bladder) and tumor mutational burdens. For example, the sub-study analyzed four prostate cancer samples with two samples from stage two prostate cancer and two samples from stage three prostate cancer.

TABLE 2

| Primary | Clinical Stage | | | | |
|---|---|---|---|---|---|
| Diagnosis | I | II | III | IV | Total |
| Breast | 0 | 1 | 1 | 0 | 2 |
| CRC | 1 | 1 | 3 | 3 | 8 |
| Melanoma | 0 | 0 | 2 | 2 | 4 |
| Bladder | 0 | 0 | 2 | 0 | 2 |
| Prostate | 0 | 2 | 2 | 0 | 4 |
| Total | 1 | 4 | 10 | 5 | 20 |

Based on the data from the samples shown in Table 2, FIG. 6 illustrates the results of sub-study that determined the residual tumor material likelihood threshold 602 for determining the presence of residual tumor material (e.g., MRD positivity). As shown in FIG. 6, a residual tumor material graph 600 shows the sub-study assessing 20 initial samples against a panel of 30 normal plasma samples depicted as dots in the residual tumor material graph 600. For example, the sub-study generated 20 tumor profiles 604 from various tumor or cancer types with differing total mutational burden levels and determined a residual tumor material likelihood (RTML) score 606 shown along the y-axis for the tumor profiles 604 by assessing the tumor profiles 604 across the panel of 30 normal plasma samples. Based on the RTML score 606 shown along the y-axis for the tumor profiles 604, the MRD system 106 set a residual tumor material likelihood threshold 602 of 7 with a specificity (e.g., the proportion of tumor profiles given correct presence of residual tumor material) of 99.7%. In some cases, the MRD system 106 determines the residual tumor material likelihood threshold 602 based on the level of specificity. As further shown in FIG. 6, if the residual tumor likelihood exceeds the residual tumor material likelihood threshold 602, the subject has detectable ctDNA indicating the presence of the tumor in the subject. Alternatively, if the residual tumor likelihood falls below the residual tumor material likelihood threshold 602, the subject has an indication of the absence of the tumor.

In one or more embodiments, the MRD system 106 can provide for display on a client device the level of residual tumor material, presence or absence of residual tumor, and/or the residual tumor material likelihood threshold 602. By implementing an MRD model as described above, the MRD system 106 more accurately identifies and removes noisy nucleotide reads. Indeed, by using the MRD models as described above to generate likelihoods of residual tumor material, the MRD system 106 can identify noisy nucleotide reads that may falsely indicate the presence of ctDNA within subsequent samples.

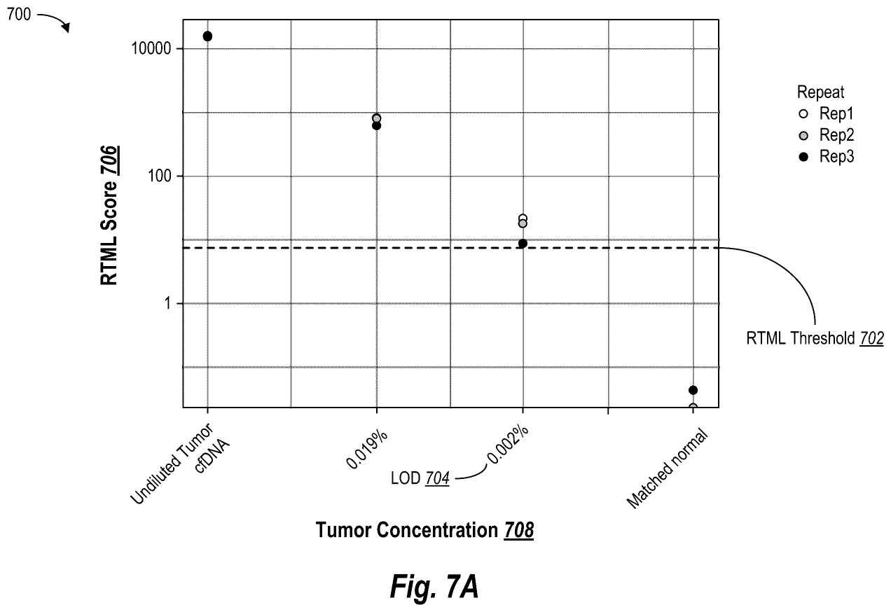

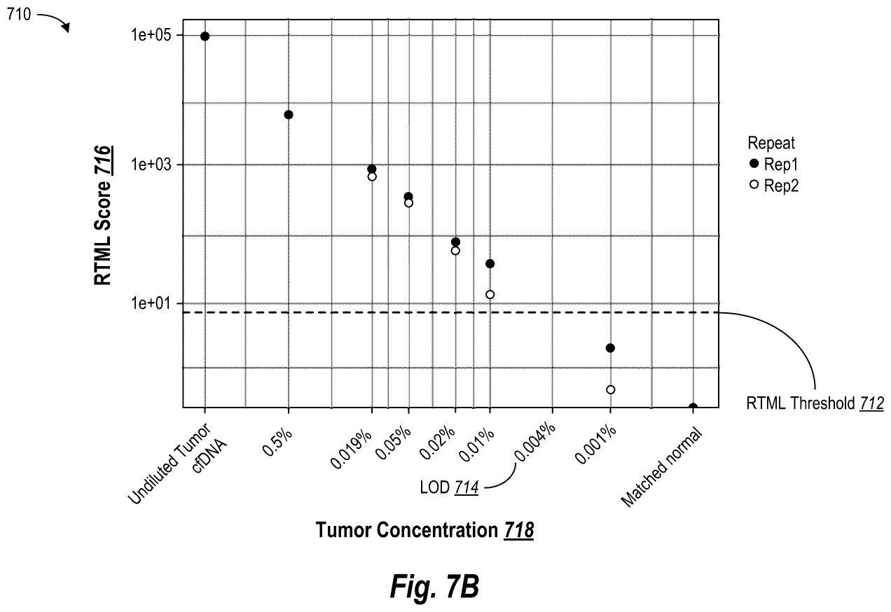

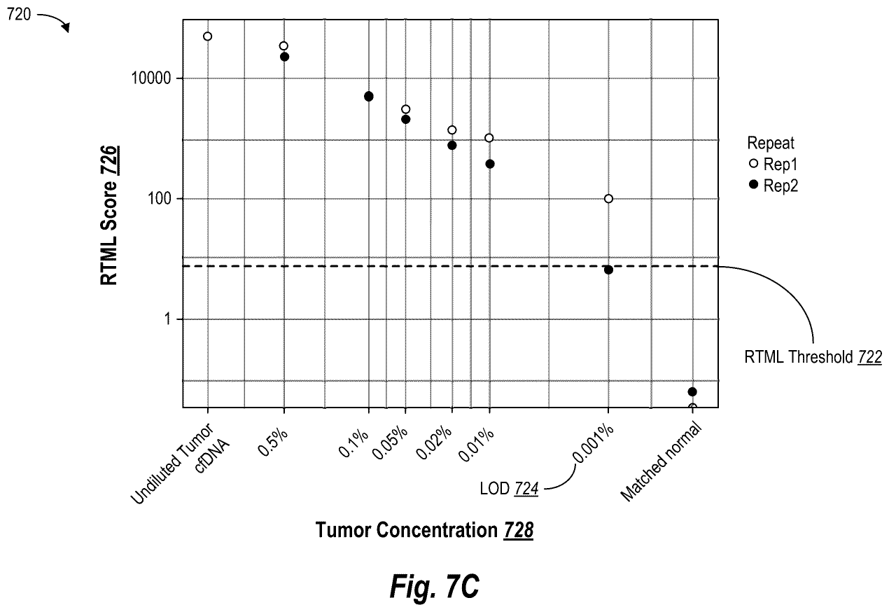

In accordance with one or more embodiments, FIGS. 7A-7C illustrate the accuracy and sensitivity of the MRD system 106. For example, FIGS. 7A-7C illustrate graphs showing experimental results of the MRD system 106 using dilutions of SeraCare ctDNA (e.g., the control sample) and ctDNA positive plasma samples. In particular, a graph 700 in FIG. 7A shows results for ctDNA within the control sample; and graphs 710 and 720 in FIGS. 7B and 7C, respectively, shows results for ctDNA within plasma samples of corresponding subjects. Additionally, each of the graphs 700, 710, and 720 depict thresholds (labeled in FIGS. 7A-7C as “RTML Threshold”) from among RTML scores 706, 716, and 726, respectively, along the corresponding y-axis. The RTML thresholds 702, 712, and 722 accordingly indicate RTML scores above which a sample is called MRD positive and accordingly indicate a limit of detection 704, 714, and 724 (labeled in FIGS. 7A-7C as “LOD”) that are determined for variations of ctDNA within the sample (e.g., plasma sample) from a corresponding subject or the control sample. As indicated by the x-axis of each of the graphs 700, 710, and 720, the variations of the samples can represent tumor concentration 708, 718, and 728 for the samples. In particular, the x-axis indicates the concentration of tumor cfDNA (e.g., expected VAF) within the sample. The x-axis of each of the graphs 700, 710, and 720 is also marked with indicators for an undiluted variation (e.g., concentration) of the control sample and the plasma samples (e.g., undiluted tumor cfDNA on the far left) and variations resulting from different levels of dilution (with positions farther to the right indicating more dilution and/or a lower expected VAF).

As shown in the graphs 700, 710, and 720, the location of the replicates (e.g., dots) along the x-axis for tumor concentration 708, 718, and 728 indicates the LOD 704, 714, and 724, respectively, for the control sample and the plasma samples of the corresponding graph. In particular, the intersection between the location of the replicates along the corresponding x-axis for tumor concentration 708, 718, and 728 and the RTML thresholds 702, 712, and 722 indicates the LOD 704, 714, and 724, respectively, for the tumor concentration 708, 718, and 728 of the control sample and the plasma samples. For example, as shown in FIG. 7A, the LOD 704 of the diluted control sample is 0.002%. As shown in FIGS. 7A-7C, the increasing dilution of the concentration of ctDNA for the control sample and plasma samples decreased the LOD 704, 714, and 724. In some cases, the diluted variations correspond to variations in which the tumor mutational burden has been diluted.

As further indicated by FIGS. 7A-7C, the linear trend and tightness of the replicates in FIGS. 7A-7C demonstrates the reproducibility and accuracy of the MRD system 106. In particular, the similar trend between the serial dilutions of the control sample in FIG. 7A and the plasma samples in FIGS. 7B and 7C demonstrates that the MRD system 106 can accurately detect residual tumor material (when present) in corresponding subjects.

Along a corresponding y-axis, each of the graphs 700, 710, and 720 shows the RTML scores 706, 716, and 726 for the control sample and the plasma sample of the corresponding subject. As further indicated in each of the graphs 700, 710, and 720, the MRD system 106 compares (i) the RTML scores 706, 716, and 726 shown along the corresponding y-axis and determined for the control sample and the plasma sample of the corresponding subject to (ii) the RTML threshold 702, 712, and 722 determined for a matched normal sample of the corresponding subject. As shown by the LOD 704, 714, and 724, the MRD system 106 can achieve detection of residual tumor material down to 0.001% target VAF. Indeed, in some cas+es, the MRD system 106 determines that each of the sample variations above the LOD 704, 714, and 724 have the presence of a tumor and each of the sample variations below the horizontal lines do not have a presence of the tumor (i.e., normal).

As further shown by the graphs 700, 710, and 720 of FIGS. 7A-7C, the MRD system 106 produces the RTML scores 706, 716, and 726 shown along the y-axis for the undiluted sample variations of each subject and the control sample that are well above the corresponding horizontal line, providing a clear indication that these sample variations are MRD positive. Additionally, as the tumor burden with the samples is reduced, the RTML scores 706, 716, and 726 lower along the y-axis of the graphs 700, 710, and 720, respectively, showing that the MRD system 106 is responsive to the level of tumor burden within a sample.

In addition or in the alternative to improving the accuracy and sensitivity of MRD detection, in some embodiments, the present disclosure further includes one or more embodiments in which the MRD system 106 performs or facilitates methods of treating cancer or other forms of MRD in a subject. Such methods may comprise obtaining a biological sample (e.g., a FFPE tissue sample) of a cancer from the subject before the subject is administered a first anti-cancer therapy; detecting one or more variants (e.g., a subset of variants) in the biological sample to generate a tumor profile for the subject before the subject is administered the first anti-cancer therapy; administering the first anti-cancer therapy to the subject; obtaining a liquid biopsy from the subject after the subject was administered the first anti-cancer therapy; detecting the one or more variants in the liquid biopsy, wherein the cancer has recurred and wherein the one or more variants detected in the liquid biopsy are present in the tumor profile; and administering a second anti-cancer therapy to the subject after recurrence of the cancer.

In some embodiments, “treating” or “treatment” of a disease, disorder, or condition includes, at least partially, (1) preventing the disease, disorder, or condition, e.g., causing the clinical symptoms of the disease, disorder, or condition not to develop in a mammal that is exposed to or predisposed to the disease, disorder, or condition but does not yet experience or display symptoms of the disease, disorder, or condition; (2) inhibiting the disease, disorder, or condition, e.g., arresting or reducing the development of the disease, disorder, or condition or its clinical symptoms; or (3) relieving the disease, disorder, or condition, e.g., causing regression of the disease, disorder, or condition or its clinical symptoms. The treating or treatment of a disease or disorder may include treating or the treatment of cancer.

The term “treating cancer” refers to administration to a mammal afflicted with a cancerous condition and refers to an effect that alleviates the cancerous condition by killing the cancerous cells, but also to an effect that results in the inhibition of growth and/or metastasis of the cancer.

The anti-cancer therapy (e.g., first anti-cancer therapy or second anti-cancer therapy) can include any well-known therapies to treat cancer, including, but not limited to, surgical removal of the cancer, administration of chemotherapy, administration of radiation, administration of antibody therapies, and administration of anti-cancer drugs. In some embodiments, the second anti-cancer therapy is different than the first anti-cancer therapy.

The term “chemotherapy” refers to the treatment of cancer or a disease or disorder caused by a virus, bacterium, other microorganism, or an inappropriate immune response using specific chemical agents, drugs, or radioactive agents that are selectively toxic and destructive to malignant cells and tissues, viruses, bacteria, or other microorganisms. Chemotherapeutic agents or drugs, such as an anti-folate (e.g., Methotrexate) or any other agent or drug useful in treating cancer, an inflammatory disease, or an autoimmune disease are preferred. Suitable chemotherapeutic agents and drugs include, but are not limited to, actinomycin D, adriamycin, altretamine, azathioprine, bleomycin, busulphan, capecitabine, carboplatin, carmustine, chlorambucil, cisplatin, cladribine, crisantaspase, cyclophosphamide, cytarabine, dacarbazine, daunorubicin, doxorubicin, epirubicin, etoposide, fludarabine, fluorouracil, gemcitabine, hydroxyurea, idarubicin, ifosfamide, irinotecan, liposomal doxorubicin, lomustine, melphalan, mercaptopurine, Methotrexate, mitomycin, mitozantrone, oxaliplatin, paclitaxel, pentostatin, procarbazine, raltitrexed, steroids, streptozocin, taxol, taxotere, temozolomide, thioguanine, thiotepa, tomudex, topotecan, treosulfan, uft (uracil-tegufur), vinblastine, vincristine, vindesine, and vinorelbine.

The present disclosure further includes one or more embodiments in which the MRD system 106 performs or facilitates methods for administering an anti-cancer therapy to a subject with cancer. Such methods may comprise obtaining a liquid biopsy from a subject after a predetermined period of time and after the subject was administered a first anti-cancer therapy (e.g., after the subject's cancer has gone into remission); detecting one or more variants (e.g., a subset of variants) in the liquid biopsy, wherein recurrence of the cancer has occurred and wherein the one or more variants detected in the liquid biopsy are present in a tumor profile created from information obtained from the subject prior to treatment with the first anti-cancer therapy; and administering a second anti-cancer therapy to the subject after recurrence of the cancer is detected.

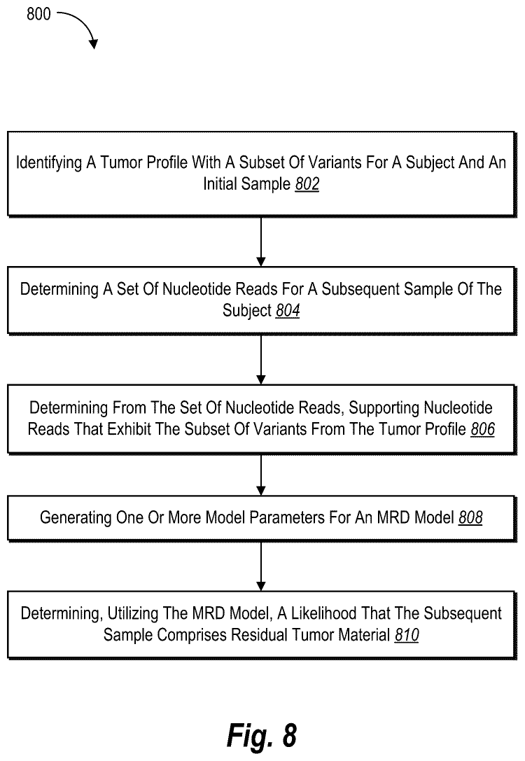

FIGS. 1-7C, the corresponding text and the examples provide a number of different methods, systems, devices, and non-transitory computer-readable media of the MRD system 106. In addition to the foregoing, one or more embodiments can also be described in terms of flowcharts comprising acts for accomplishing particular results, as shown in FIG. 8. FIG. 8 may be performed with more or fewer acts. Further, the acts may be performed in different orders. Additionally, the acts described herein may be repeated or performed in parallel with one another or in parallel with different instances of the same or similar acts.

FIG. 8 illustrates a flowchart of a series of acts 800 for determining a likelihood that the subsequent sample comprises residual tumor material in accordance with one or more embodiments. While FIG. 8 illustrates acts according to one embodiment, alternative embodiments may omit, add to, reorder, and/or modify any of the acts shown in FIG. 8. In some implementations, the acts of FIG. 8 are performed as part of a method. In some instances, a non-transitory computer-readable medium stores instructions thereon that, when executed by at least one processor, cause a computing device to perform the acts of FIG. 8. In some implementations, a system performs the acts of FIG. 8. For example, in one or more cases, a system includes at least one processor and a non-transitory computer readable medium comprising instructions that, when executed by the at least one processor, cause the system to perform the acts of FIG. 8.

As shown in FIG. 8, the series of acts 800 includes an act 802 of identifying a tumor profile with a subset of variants for a subject and an initial sample, an act 804 of determining a set of nucleotide reads for a subsequent sample of the subject, an act 806 of determining from the set of nucleotide reads, supporting nucleotide reads that exhibit the subset of variants from the tumor profile, an act 808 of generating one or more model parameters for an MRD model, and an act 810 of determining, utilizing the MRD model, a likelihood that the subsequent sample comprises residual tumor material. For example, the series of acts 800 can include acts to perform any of the operations described in the following clauses:

CLAUSE 1. A computer-implemented method comprising:
identifying, for a subject and an initial sample comprising a tumor, a tumor profile comprising a subset of variants within target genomic regions;
determining, for a subsequent sample of the subject, a set of nucleotide reads across the target genomic regions;
determining, from the set of nucleotide reads for the subsequent sample of the subject, supporting nucleotide reads that exhibit, within the target genomic regions, one or more variants of the subset of variants from the tumor profile;
generating, for a minimal residual disease (MRD) model, one or more model parameters indicating a presence of the tumor within the subject; and determining, utilizing the MRD model and the one or more model parameters, a likelihood that the subsequent sample comprises residual tumor material based on the supporting nucleotide reads for the subsequent sample.

CLAUSE 2. The computer-implemented method of clause 1, further comprising:
determining the supporting nucleotide reads by determining a count of supporting nucleotide reads for the subsequent sample exhibiting target single nucleotide variants (SNVs) at genomic coordinates from the tumor profile; and
determining, utilizing the MRD model and the one or more model parameters, the likelihood that the subject comprises the residual tumor material based on the count of supporting nucleotide reads exhibiting the target SNVs.

CLAUSE 3. The computer-implemented method of clause 1, further comprising:
determining the supporting nucleotide reads by determining counts of nucleotide reads for the subsequent sample that map to copy number variation (CNV) segments within a genome; and determining, utilizing the MRD model and the one or more model parameters, the likelihood that the subject comprises the residual tumor material based on the counts of supporting nucleotide reads mapping to the CNV segments.

CLAUSE 4. The computer-implemented method of clause 3, further comprising determining, utilizing the MRD model and the one or more model parameters, the likelihood that the subject comprises the residual tumor material by:
determining, for each frequency of a range of sample-wide tumor allele frequencies, a first likelihood of the presence of the tumor given a sample-wide tumor allele frequency;
determining, for each frequency of the range of sample-wide tumor allele frequencies, a second likelihood of an absence of the tumor given the sample-wide tumor allele frequency; and
determining a likelihood that the subsequent sample comprises the residual tumor material within the CNV segments based on the first likelihood of the presence of the tumor and the second likelihood of the absence of the tumor for each frequency of the range of sample-wide tumor allele frequencies.

CLAUSE 5. The computer-implemented method of clause 1, further comprising determining the supporting nucleotide reads by:
determining a count of supporting nucleotide reads for the subsequent sample exhibiting target structural variants (SVs) from the tumor profile; and
determining, utilizing the MRD model and the one or more model parameters, the likelihood that the subject comprises the residual tumor material based on the count of supporting nucleotide reads exhibiting the target SVs.

CLAUSE 6. The computer-implemented method of clause 1, wherein the initial sample comprises a sample or the subsequent sample from tumor cells of the subject and the subsequent sample comprises a plasma sample comprising cell-free deoxyribonucleic acid (cfDNA).

CLAUSE 7. The computer-implemented method of clause 1, wherein the initial sample or the subsequent sample comprises a bone marrow sample, a urine sample, a saliva sample, a stool sample, or a bile sample comprising cell-free deoxyribonucleic acid (cfDNA).

CLAUSE 8. The computer-implemented method of clause 1, further comprising:
determining, from the set of nucleotide reads for the subsequent sample of the subject, additional supporting nucleotide reads that exhibit, within the target genomic regions, an additional type of variants from the subset of variants from the tumor profile;
generating, for an additional MRD model, additional one or more model parameters indicating a presence of the tumor within the subject; and
determining, utilizing the additional MRD model and the additional one or more model parameters, an additional likelihood that the subsequent sample comprises the residual tumor material based on the additional supporting nucleotide reads for the subsequent sample.

CLAUSE 9. The computer-implemented method of clause 8, further comprising:
combining the likelihood that the subsequent sample comprises the residual tumor material based on the supporting nucleotide reads and the additional likelihood that the subsequent sample comprises the residual tumor material based on the additional supporting nucleotide reads; and
generating a posterior probability that the subsequent sample comprises the residual tumor material based on the combination of the likelihood and the additional likelihood.

CLAUSE 10. The computer-implemented method of clause 8, further comprising determining a presence or absence of the tumor within the subject based on the additional likelihood that the subsequent sample comprises the residual tumor material.

CLAUSE 11. The computer-implemented method of clause 1, further comprising determining a presence or absence of the tumor within the subject based on the likelihood that the subsequent sample comprises the residual tumor material.

CLAUSE 12. The computer-implemented method of clause 1, further comprising determining the supporting nucleotide reads that exhibit one or more variants of the subset of variants from the tumor profile by:
determining a count of supporting nucleotide reads for the subsequent sample exhibiting one or more variants of the subset of variants from the tumor profile;
determining a count of additional supporting nucleotide reads for the subsequent sample exhibiting an additional subset of variants from a normal sample of the subject; and
removing the count of additional supporting nucleotide reads exhibiting one or more variants of the subset of variants from the count of supporting nucleotide reads to determine a filtered count of supporting nucleotide reads for the subsequent sample exhibiting one or more variants of the subset of variants from the tumor profile.

CLAUSE 13. The computer-implemented method of clause 1, wherein the subset of variants within the target genomic regions for the tumor profile are determined using whole genome sequencing, whole exome sequencing, or a targeted assay.

CLAUSE 14. The computer-implemented method of clause 1, further comprising identifying the tumor profile by:
identifying a subject-specific tumor profile comprising a first subset of variants within the target genomic regions, wherein the first subset of variants are specific to the subject; or
identifying a generic tumor profile comprising a second subset of variants within the target genomic regions, wherein the second subset of variants are specific to the tumor.

CLAUSE 15. The computer-implemented method of clause 1, further comprising:
determining a residual tumor material likelihood threshold; and
determining the presence of the residual tumor material based on the likelihood that the subsequent sample comprises residual tumor material exceeding the residual tumor material likelihood threshold.

CLAUSE 16. The computer-implemented method of clause 1, further comprising:
identifying or determining an anti-cancer therapy for the subject; or
administering the anti-cancer therapy to the subject.

CLAUSE 17. A computer-implemented method comprising:
obtaining a biological sample of a cancer from the subject before the subject is administered a first anti-cancer therapy;
identifying, for the subject and the biological sample of the cancer, a tumor profile comprising a subset of variants within target genomic regions, wherein the tumor profile is identified before the subject is administered the first anti-cancer therapy;
administering the first anti-cancer therapy to the subject;
obtaining a liquid biopsy from the subject after the subject is administered the first anti-cancer therapy;
detecting one or more variants of the subset of variants in the liquid biopsy, wherein the cancer has recurred and wherein the one or more variants detected in the liquid biopsy are present in the subset of variants of the tumor profile; and
administering a second anti-cancer therapy to the subject after recurrence of the cancer.

CLAUSE 18. A computer-implemented method comprising:
obtaining a liquid biopsy from a subject after a predetermined period of time and after the subject was administered a first anti-cancer therapy;
identifying, for the subject and the liquid biopsy, a tumor profile comprising a subset of variants within target genomic regions, wherein the tumor profile is identified before the subject is administered the first anti-cancer therapy; and
administering a second anti-cancer therapy to the subject after recurrence of the cancer is detected.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Implementations in which images are obtained in different color channels, for example, coinciding with different labels used to distinguish one nucleobase type from another are particularly applicable. In some implementations, the process to determine the nucleotide sequence of a target nucleic acid (i.e., a nucleic-acid polymer) can be an automated process. Preferred implementations include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as the release of pyrophosphate; or the like. In implementations, where two or more different nucleotides are present in a sequencing reagent, the different nucleotides can be distinguishable from each other, or alternatively, the two or more different labels can be indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred implementations include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281 (5375), 363; U.S. Pat. Nos. 6,210,891; 6,258,568 and 6,274,320, the disclosures of which are incorporated herein by reference in their entireties). In pyrosequencing, released Ppi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to the incorporation of nucleotides at the features of the array. An image can be obtained after the array is treated with a particular nucleotide type (e.g., A, T, C, or G). Images obtained after the addition of each nucleotide type will differ with regard to which features in the array are detected. These differences in the image reflect the different sequence content of the features on the array. However, the relative locations of each feature will remain unchanged in the images. The images can be stored, processed, and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.) and is also described in WO 91/06678 and WO 07/123,744, each of which is incorporated herein by reference. The availability of fluorescently-labeled terminators in which both the termination can be reversed, and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing implementations, the labels do not substantially inhibit extension under SBS reaction conditions. However, the detection labels can be removable, for example, by cleavage or degradation. Images can be captured following the incorporation of labels into arrayed nucleic acid features. In particular implementations, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such implementations, each image will show nucleic acid features that have incorporated nucleotides of a particular type. Different features are present or absent in the different images due to the different sequence content of each feature. However, the relative position of the features will remain unchanged in the images. Images obtained from such reversible terminator-SBS methods can be stored, processed, and analyzed as set forth herein. Following the image capture step, labels can be removed, and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular implementations, some or all of the nucleotide monomers can include reversible terminators. In such implementations, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30-second exposure to long-wavelength UV light. Thus, either disulfide reduction or photocleavage can be used as a cleavable linker. Another approach to reversible termination is the use of natural termination that ensues after the placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100800, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some implementations can utilize the detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes an apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on the presence of their respective signals and incorporation of the fourth nucleotide type into the nucleic acid can be determined based on the absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary implementation that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some implementations can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due to the different sequence content of each feature, but the relative position of the features will remain unchanged in the images. Images obtained from ligation-based sequencing methods can be stored, processed, and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some implementations can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such implementations, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed, and analyzed as set forth herein. In particular, the data can be treated as an image in accordance with the exemplary treatment of optical images and other images that is set forth herein.

Some implementations can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed, and analyzed as set forth herein.

Some SBS implementations include the detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular implementations, different target nucleic acids can be treated in a common reaction vessel or on a surface of a particular substrate. This allows convenient delivery of sequencing reagents, removal of unreacted reagents, and detection of incorporation events in a multiplex manner. In implementations using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle, or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as bridge amplification or emulsion PCR as described in further detail below.

The methods set forth herein can use arrays having features at any of a variety of densities including, for example, at least about 10 features/cm2, 100 features/cm2, 500 features/cm2, 1,000 features/cm2, 5,000 features/cm2, 10,000 features/cm2, 50,000 features/cm2, 100,000 features/cm2, 1,000,000 features/cm2, 5,000,000 features/cm2, or higher.

An advantage of the methods set forth herein is that they provide for rapid and efficient detection of a plurality of target nucleic acid in parallel. Accordingly, the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines, and the like. A flow cell can be configured and/or used in an integrated system for the detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing implementation as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference. The sequencing system described above sequences nucleic-acid polymers present in samples received by a sequencing device, as described further above.

Further, the methods and compositions disclosed herein may be useful to amplify a nucleic acid sample having low-quality nucleic acid molecules, such as degraded and/or fragmented genomic DNA from a forensic sample. In one implementation, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example, derived from a buccal swab, paper, fabric, or other substrates that may be impregnated with saliva, blood, or other bodily fluids. As such, in some implementations, the nucleic acid sample may comprise low amounts of, or fragmented portions of DNA, such as genomic DNA. In some implementations, target sequences can be present in one or more bodily fluids including but not limited to, blood, sputum, plasma, semen, urine, and serum. In some implementations, target sequences can be obtained from hair, skin, tissue samples, autopsy or remains of a victim. In some implementations, nucleic acids including one or more target sequences can be obtained from a deceased animal or human. In some implementations, target sequences can include nucleic acids obtained from non-human DNA such a microbial, plant, or entomological DNA. In some implementations, target sequences or amplified target sequences are directed to purposes of human identification. In some implementations, the disclosure relates generally to methods for identifying characteristics of a forensic sample. In some implementations, the disclosure relates generally to human identification methods using one or more target-specific primers disclosed herein or one or more target-specific primers designed using the primer design criteria outlined herein. In one implementation, a forensic or human identification sample containing at least one target sequence can be amplified using any one or more of the target-specific primers disclosed herein or using the primer criteria outlined herein.

The components of the MRD system 106 can include software, hardware, or both. For example, the components of the MRD system 106 can include one or more instructions stored on a computer-readable storage medium and executable by processors of one or more computing devices (e.g., the client device 108). When executed by the one or more processors, the computer-executable instructions of the MRD system 106 can cause the computing devices to perform the structural variant detection methods described herein. Alternatively, the components of the MRD system 106 can comprise hardware, such as special-purpose processing devices to perform a certain function or group of functions. Additionally, or in the alternative, the components of the MRD system 106 can include a combination of computer-executable instructions and hardware.

Furthermore, the components of the MRD system 106 performing the functions described herein with respect to the MRD system 106 may, for example, be implemented as part of a stand-alone application, as a module of an application, as a plug-in for applications, as a library function or functions that may be called by other applications, and/or as a cloud-computing model. Thus, components of the MRD system 106 may be implemented as part of a stand-alone application on a personal computing device or a mobile device. Additionally, or alternatively, the components of the MRD system 106 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Implementations of the present disclosure may comprise or utilize a special purpose or general-purpose computer including computer hardware, such as, for example, one or more processors and system memory, as discussed in greater detail below. Implementations within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general-purpose or special-purpose computer system. Computer-readable media that store computer-executable instructions are non-transitory computer-readable storage media (devices). Computer-readable media that carry computer-executable instructions are transmission media. Thus, by way of example, and not limitation, implementations of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid-state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general-purpose or special-purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links that can be used to carry desired program code means in the form of computer-executable instructions or data structures and which can be accessed by a general-purpose or special-purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. In some implementations, computer-executable instructions are executed on a general-purpose computer to turn the general-purpose computer into a special-purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Implementations of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 9 illustrates a block diagram of a computing device 900 that may be configured to perform one or more of the processes described above. One will appreciate that one or more computing devices such as the computing device 900 may implement the MRD system 106 and the sequencing system 116. As shown by FIG. 9, the computing device 900 can comprise a processor 902, a memory 904, a storage device 906, an I/O interface 908 and a communication interface 910, which may be communicatively coupled by way of a communication infrastructure 912. In certain implementations, the computing device 900 can include fewer or more components than those shown in FIG. 9. The following paragraphs describe components of the computing device 900 shown in FIG. 9 in additional detail.

In one or more implementations, the processor 902 includes hardware for executing instructions, such as those making up a computer program. As an example, and not by way of limitation, to execute instructions for dynamically modifying workflows, the processor 902 may retrieve (or fetch) the instructions from an internal register, an internal cache, the memory 904, or the storage device 906 and decode and execute them. The memory 904 may be a volatile or non-volatile memory used for storing data, metadata, and programs for execution by the processor(s). The storage device 906 includes storage, such as a hard disk, flash disk drive, or another digital storage device, for storing data or instructions for performing the methods described herein.

The I/O interface 908 allows a user to provide input to, receive output from, and otherwise transfer data to and receive data from computing device 900. The I/O interface 908 may include a mouse, a keypad or a keyboard, a touch screen, a camera, an optical scanner, network interface, modem, other known I/O devices, or a combination of such I/O interfaces. The I/O interface 908 may include one or more devices for presenting output to a user, including, but not limited to, a graphics engine, a display (e.g., a display screen), one or more output drivers (e.g., display drivers), one or more audio speakers, and one or more audio drivers. In certain implementations, the I/O interface 908 is configured to provide graphical data to a display for presentation to a user. The graphical data may be representative of one or more graphical user interfaces and/or any other graphical content as may serve a particular implementation.

The communication interface 910 can include hardware, software, or both. In any event, the communication interface 910 can provide one or more interfaces for communication (such as, for example, packet-based communication) between the computing device 900 and one or more other computing devices or networks. As an example, and not by way of limitation, the communication interface 910 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 910 may facilitate communications with various types of wired or wireless networks. The communication interface 910 may also facilitate communications using various communication protocols. The communication infrastructure 912 may also include hardware, software, or both that couples components of the computing device 900 to each other. For example, the communication interface 910 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary implementations thereof. Various implementations and aspects of the present disclosure(s) are described with reference to details discussed herein, and the accompanying drawings illustrate the various implementations. The description above and drawings are illustrative of the disclosure and are not to be construed as limiting the disclosure. Numerous specific details are described to provide a thorough understanding of various implementations of the present disclosure.

The present disclosure may be embodied in other specific forms without departing from its spirit or essential characteristics. The described implementations are to be considered in all respects only as illustrative and not restrictive. For example, the methods described herein may be performed with fewer or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

What is claimed is:

1. A system comprising:
at least one processor; and
a non-transitory computer-readable medium storing instructions that, when executed by the at least one processor, cause the system to:
identify, for a subject and an initial sample comprising a tumor, a tumor profile comprising a subset of variants within target genomic regions;
determine, for a subsequent sample of the subject, a set of nucleotide reads across the target genomic regions;
determine, from the set of nucleotide reads for the subsequent sample of the subject, supporting nucleotide reads that exhibit, within the target genomic regions, one or more variants of the subset of variants from the tumor profile;
generate, for a minimal residual disease (MRD) model, one or more model parameters indicating a presence of the tumor within the subject; and
determine, utilizing the MRD model and the one or more model parameters, a likelihood that the subsequent sample comprises residual tumor material based on the supporting nucleotide reads for the subsequent sample.

2. The system of claim 1, further comprising instructions that, when executed by the at least one processor, cause the system to:
determine the supporting nucleotide reads by determining a count of supporting nucleotide reads for the subsequent sample exhibiting target single nucleotide variants (SNVs) at genomic coordinates from the tumor profile; and
determine, utilizing the MRD model and the one or more model parameters, the likelihood that the subject comprises the residual tumor material based on the count of supporting nucleotide reads exhibiting the target SNVs.

3. The system of claim 1, further comprising instructions that, when executed by the at least one processor, cause the system to:
determine the supporting nucleotide reads by determining counts of nucleotide reads for the subsequent sample that map to copy number variation (CNV) segments within a genome; and
determine, utilizing the MRD model and the one or more model parameters, the likelihood that the subject comprises the residual tumor material based on the counts of supporting nucleotide reads mapping to the CNV segments.

4. The system of claim 3, further comprising instructions that, when executed by the at least one processor, cause the system to determine, utilizing the MRD model and the one or more model parameters, the likelihood that the subject comprises the residual tumor material by:
determining, for each frequency of a range of sample-wide tumor allele frequencies, a first likelihood of the presence of the tumor given a sample-wide tumor allele frequency;
determining, for each frequency of the range of sample-wide tumor allele frequencies, a second likelihood of an absence of the tumor given the sample-wide tumor allele frequency; and
determining a likelihood that the subsequent sample comprises the residual tumor material within the CNV segments based on the first likelihood of the presence of the tumor and the second likelihood of the absence of the tumor for each frequency of the range of sample-wide tumor allele frequencies.

5. The system of claim 1, further comprising instructions that, when executed by the at least one processor, cause the system to:
determine the supporting nucleotide reads by determining a count of supporting nucleotide reads for the subsequent sample exhibiting target structural variants (SVs) from the tumor profile; and
determine, utilizing the MRD model and the one or more model parameters, the likelihood that the subject comprises the residual tumor material based on the count of supporting nucleotide reads exhibiting the target SVs.

6. The system of claim 1, wherein the initial sample or the subsequent sample comprises a sample from tumor cells of the subject and the subsequent sample comprises a plasma sample comprising cell-free deoxyribonucleic acid (cfDNA).

7. The system of claim 1, wherein the initial sample or the subsequent sample comprises a bone marrow sample, a urine sample, a saliva sample, a stool sample, or a bile sample comprising cell-free deoxyribonucleic acid (cfDNA).

8. The system of claim 1, further comprising instructions that, when executed by the at least one processor, cause the system to:
determine, from the set of nucleotide reads for the subsequent sample of the subject, additional supporting nucleotide reads that exhibit, within the target genomic regions, an additional type of variants from the subset of variants from the tumor profile;
generate, for an additional MRD model, additional one or more model parameters indicating a presence of the tumor within the subject; and
determine, utilizing the additional MRD model and the additional one or more model parameters, an additional likelihood that the subsequent sample comprises the residual tumor material based on the additional supporting nucleotide reads for the subsequent sample.

9. The system of claim 8, further comprising instructions that, when executed by the at least one processor, cause the system to:
combine the likelihood that the subsequent sample comprises the residual tumor material based on the supporting nucleotide reads and the additional likelihood that the subsequent sample comprises the residual tumor material based on the additional supporting nucleotide reads; and
generate a posterior probability that the subsequent sample comprises the residual tumor material based on the combination of the likelihood and the additional likelihood.

10. The system of claim 8, further comprising instructions that, when executed by the at least one processor, cause the system to determine a presence or absence of the tumor within the subject based on the additional likelihood that the subsequent sample comprises the residual tumor material.

11. A non-transitory computer-readable medium storing instructions that, when executed by at least one processor, cause a system to:
identify, for a subject and an initial sample comprising a tumor, a tumor profile comprising a subset of variants within target genomic regions;
determine, for a subsequent sample of the subject, a set of nucleotide reads across the target genomic regions;
determine, from the set of nucleotide reads for the subsequent sample of the subject, supporting nucleotide reads that exhibit, within the target genomic regions, one or more variants of the subset of variants from the tumor profile;
generate, for a minimal residual disease (MRD) model, one or more model parameters indicating a presence of the tumor within the subject; and
determine, utilizing the MRD model and the one or more model parameters, a likelihood that the subsequent sample comprises residual tumor material based on the supporting nucleotide reads for the subsequent sample.

12. The non-transitory computer-readable medium of claim 11, further comprising instructions that, when executed by the at least one processor, cause the system to determine a presence or absence of the tumor within the subject based on the likelihood that the subsequent sample comprises the residual tumor material.

13. The non-transitory computer-readable medium of claim 11, further comprising instructions that, when executed by the at least one processor, cause the system to determine the supporting nucleotide reads that exhibit one or more variants of the subset of variants from the tumor profile by:
determining a count of supporting nucleotide reads for the subsequent sample exhibiting one or more variants of the subset of variants from the tumor profile;
determining a count of additional supporting nucleotide reads for the subsequent sample exhibiting an additional subset of variants from a normal sample of the subject; and
removing the count of additional supporting nucleotide reads exhibiting one or more variants of the subset of variants from the count of supporting nucleotide reads to determine a filtered count of supporting nucleotide reads for the subsequent sample exhibiting one or more variants of the subset of variants from the tumor profile.

14. The non-transitory computer-readable medium of claim 11, wherein the subset of variants within the target genomic regions for the tumor profile are determined using whole genome sequencing, whole exome sequencing, or a targeted assay.

15. The non-transitory computer-readable medium of claim 11, further comprising instructions that, when executed by the at least one processor, cause the system to identify the tumor profile by:
identifying a subject-specific tumor profile comprising a first subset of variants within the target genomic regions, wherein the first subset of variants are specific to the subject; or
identifying a generic tumor profile comprising a second subset of variants within the target genomic regions, wherein the second subset of variants are specific to the tumor.

16. The system of claim 1, further comprising instructions that, when executed by the at least one processor, cause the system to:
determine a residual tumor material likelihood threshold; and
determine the presence of the residual tumor material based on the likelihood that the subsequent sample comprises residual tumor material exceeding the residual tumor material likelihood threshold.

17. A computer-implemented method comprising:
identifying, for a subject and an initial sample comprising a tumor, a tumor profile comprising a subset of variants within target genomic regions;
determining, for a subsequent sample of the subject, a set of nucleotide reads across the target genomic regions;
determining, from the set of nucleotide reads for the subsequent sample of the subject, supporting nucleotide reads that exhibit, within the target genomic regions, one or more variants of the subset of variants from the tumor profile;
generating, for a minimal residual disease (MRD) model, one or more model parameters indicating a presence of the tumor within the subject; and
determining, utilizing the MRD model and the one or more model parameters, a likelihood that the subsequent sample comprises residual tumor material based on the supporting nucleotide reads for the subsequent sample.

18. The computer-implemented method of claim 17, wherein:
determining the supporting nucleotide reads comprises determining a count of supporting nucleotide reads for the subsequent sample exhibiting target single nucleotide variants (SNVs) at genomic coordinates from the tumor profile; and
determining the likelihood that the subject comprises the residual tumor material comprises determining, utilizing the MRD model and the one or more model parameters, the likelihood that the subject comprises the residual tumor material based on the count of supporting nucleotide reads exhibiting the target SNVs.

19. The computer-implemented method of claim 17, wherein:
determining the supporting nucleotide reads comprises determining counts of nucleotide reads for the subsequent sample that map to copy number variation (CNV) segments within a genome; and
determining the likelihood that the subject comprises the residual tumor material comprises determining, utilizing the MRD model and the one or more model parameters, the likelihood that the subject comprises the residual tumor material based on the counts of supporting nucleotide reads mapping to the CNV segments.

20. The computer-implemented method of claim 17, further comprising:
identifying or determining an anti-cancer therapy for the subject; or
administering the anti-cancer therapy to the subject.
